SEQUENCE LISTING (9 sequences)

SEQ ID NO: 1 - protein, 410 aa, synthetic construct
MDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 2 - protein, 414 aa, synthetic construct
MDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYIEPEA

SEQ ID NO: 3 - protein, 10 aa, unidentified
  VARIANT 6..10: note = up to 5 H residues can be deleted
HHHHHHHHHH

SEQ ID NO: 4 - protein, 6 aa, synthetic construct
HHHHHH

SEQ ID NO: 5 - protein, 8 aa, synthetic construct
HHHHHHHH

SEQ ID NO: 6 - protein, 4 aa, synthetic construct
EPEA

SEQ ID NO: 7 - protein, 72 aa, synthetic construct
NANPNANPNANPNANPNANPNANPNANPNANPNANPNANPNANPNANPNANPNANPNANPNANPNANPNANP

SEQ ID NO: 8 - protein, 105 aa, synthetic construct
NKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSV

SEQ ID NO: 9 - protein, 226 aa, synthetic construct
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

(19) United States
(12) Patent Application Publication
(10) Pub. No.: US 2024/0415947 A1
(43) Pub. Date: Dec. 19, 2024

(54) MALARIA VACCINE FORMULATIONS

(71) Applicants: NOVAVAX AB, Uppsala (SE); NOVAVAX, INC., Gaithersburg, MD (US)

(72) Inventors: Jenny M. REIMER, Uppsala (SE); Karin LÖVGREN BENGTSSON, Stockholm (SE); Louis FRIES

(21) Appl. No.: 18/704,632

(22) PCT Filed: Oct. 25, 2022
(86) PCT No.: PCT/US2022/078665

§ 371 Date: Apr. 25, 2024

Related U.S. Application Data
(60) Provisional application No. 63/271,420, filed on Oct. 25, 2021.
(60) Provisional application No. 63/281,332, filed on Nov. 19, 2021.

(51) Int. Cl. A61K 39/015; A61K 39/00; A61K 39/29; A61K 39/39; A61P 37/04

(52) U.S. Cl. CPC A61K 39/015; A61K 39/292; A61K 39/39; A61P 37/04; A61K 2039/545; A61K 2039/55577

(57) ABSTRACT

Disclosed herein are immunogenic compositions comprising an antigen of a Plasmodium parasite. Methods of administering the aforementioned compositions are also disclosed.

### CROSS REFERENCE TO RELATED APPLICATIONS

This application claims priority to the following applications, each of which is incorporated by reference in its entirety for all purposes: U.S. Provisional Application No. 63/271,420, filed Oct. 25, 2021 and U.S. Provisional Application No. 63/281,332, filed Nov. 19, 2021.

### REFERENCE TO AN ELECTRONIC SEQUENCE LISTING

The contents of the electronic sequence listing (NOVV_094_02WO_SeqList_ST26.xml; Size: (10,386 bytes; and Date of Creation: Oct. 25, 2022) is herein incorporated by reference in its entirety.

### FIELD OF THE DISCLOSURE

The present disclosure relates to compositions and methods for inducing immune responses against Plasmodium parasites.

### BACKGROUND OF THE INVENTION

Malaria is the leading cause of childhood mortality. As of 2019, there were an estimated 229 million cases of malaria worldwide. In 2019, 409,000 people died from malaria. There is only one approved vaccine for preventing malaria, RTS, S/AS01 (RTS,S) (MOSQUIRIX®). The protection RTS, S provides against malaria wanes over time. There is a need in the art for new vaccines and methods for preventing malaria.

### SUMMARY OF THE INVENTION

The present disclosure provides immunogenic compositions for inducing immune responses against malaria. The present disclosure also provides novel methods of administering said immunogenic compositions.

In embodiments, provided herein is an immunogenic composition comprising an antigen of a Plasmodium parasite. In embodiments, provided herein is a method of stimulating an immune response against a Plasmodium parasite in a subject comprising administering the immunogenic composition. In embodiments, the Plasmodium parasite is Plasmodium falciparum, Plasmodium malariae, Plasmodium vivax, Plasmodium ovale, or Plasmodium knowlesi. In embodiments, the antigen comprises a circumsporozoite (CS) protein or a fragment thereof. In embodiments, the CS protein is from Plasmodium falciparum. In embodiments, the antigen comprises a hepatitis B surface antigen (HBsAg) or fragment thereof. In embodiments, the antigen comprises an amino acid sequence of SEQ ID NO: 1 or an amino acid sequence that is at least 80%, at least 85%, at least 90%, at least 95%, at least 96%, at least 97%, at least 98%, at least 99%, or 100% identical to SEQ ID NO: 1. In embodiments, the immunogenic composition comprises an adjuvant. In embodiments, the adjuvant comprises at least two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. In embodiments, fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant. In embodiments, fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant. In embodiments, the adjuvant is administered at a dose of about 50 μg.

In embodiments, the method of stimulating an immune response against a Plasmodium parasite comprises administering from about 0.1 μg to about 100 μg of antigen. In embodiments, the method of stimulating an immune response against a Plasmodium parasite comprises administering from about 0.1 μg to about 10 μg of antigen. In embodiments, the method of stimulating an immune response against a Plasmodium parasite comprises administering from about 0.1 μg to about 5 μg of antigen. In embodiments, the method of stimulating an immune response against a Plasmodium parasite comprises administering from about 0.1 μg to about 3 μg of antigen. In embodiments, the method of stimulating an immune response against a Plasmodium parasite comprises administering from about 0.1 μg to about 2 μg of antigen. In embodiments, the method comprises administering a first dose and a second dose of the immunogenic composition. In embodiments, the method comprises administering a third dose of the immunogenic composition. In embodiments, the dose of antigen in the second dose is less than the dose of antigen in the first dose. In embodiments, the dose of antigen in the third dose is less than the dose of antigen in the first dose. In embodiments, the first and second dose comprise the same amount of antigen. In embodiments, the second dose and third dose comprise the same amount of antigen. In embodiments, the first dose and third dose comprise the same amount of antigen. In embodiments, the third dose comprises about 90%, about 80%, about 70%, about 60%, about 50%, about 40%, about 30%, about 20%, or about 10% of the antigen in the first dose. In embodiments, the second dose comprises about 90%, about 80%, about 70%, about 60%, about 50%, about 40%, about 30%, about 20%, or about 10% of the antigen in the first dose. In embodiments, the second dose is administered about 28 days after the first dose. In embodiments, the third dose is administered about 56 days after the first dose. In embodiments, the third dose is administered about 168 days after the first dose.

In embodiments, provided herein is a method of stimulating an immune response against a Plasmodium parasite in a subject comprising administering:
(i) a first dose of an immunogenic composition comprising an antigen of a Plasmodium parasite; wherein the first dose comprises about 10 μg of the antigen;
(ii) a second dose of the immunogenic composition, wherein the second dose comprises about 10 μg of the antigen; and
(iii) a third dose of the immunogenic composition, wherein the third dose comprises about 2 μg of the antigen.

In embodiments, the second dose is administered about 28 days after the first dose. In embodiments, the third dose is administered about 56 days after the first dose. In embodiments, the third dose is administered about 168 days after the first dose. In embodiments, the Plasmodium parasite is Plasmodium falciparum, Plasmodium malariae, Plasmodium vivax, Plasmodium ovale, or Plasmodium knowlesi. In embodiments, the antigen comprises a circumsporozoite (CS) protein or a fragment thereof. In embodiments, the CS protein is from Plasmodium falciparum. In embodiments, the antigen comprises a hepatitis B surface antigen (HBsAg) or fragment thereof. In embodiments, the antigen comprises an amino acid sequence of SEQ ID NO: 1 or an amino acid sequence that is at least 80%, at least 85%, at least 90%, at least 95%, at least 96%, at least 97%, at least 98%, at least 99%, or 100% identical to SEQ ID NO: 1. In embodiments, the immunogenic composition comprises an adjuvant. In embodiments, the adjuvant comprises at least two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. In embodiments, fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant. In embodiments, fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant. In embodiments, the adjuvant is administered at a dose of about 50 μg.

In embodiments, provided herein is a method of stimulating an immune response against a Plasmodium parasite in a subject comprising administering an immunogenic composition comprising an antigen of a Plasmodium parasite, wherein the antigen comprises a circumsporozoite (CS) protein or a fragment thereof and a hepatitis B surface antigen (HBsAg) or a fragment thereof. In embodiments, the antigen of the Plasmodium parasite comprises the amino acid sequence of SEQ ID NO: 1 or SEQ ID NO: 2 or an amino acid sequence with at least 80%, at least 85%, at least 90%, at least 91%, at least 92%, at least 93%, at least 94%, at least 95%, at least 96%, at least 97%, at least 98%, at least 99%, or at least 99.5% identity to SEQ ID NO: 1 or SEQ ID NO: 2. In embodiments, the immunogenic composition comprises an adjuvant. In embodiments, the immunogenic composition comprises from about 1 μg to about 100 μg of adjuvant. In embodiments, the immunogenic composition comprises from about 25 μg to about 75 μg of adjuvant. In embodiments, the immunogenic composition comprises about 50 μg of adjuvant. In embodiments, the immunogenic composition comprises about 25 μg of adjuvant. In embodiments, the adjuvant comprises at least two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. In embodiments, fraction A of Quillaja Saponaria Molina accounts for 50-96% by weight and fraction C of Quillaja Saponaria Molina accounts for the remainder, respectively, of the sum of the weights of fraction A of Quillaja Saponaria Molina and fraction C of Quillaja Saponaria Molina in the adjuvant. In embodiments, fraction A of Quillaja Saponaria Molina accounts for at least 75% by weight and fraction C of Quillaja Saponaria Molina accounts for the remainder, respectively, of the sum of the weights of fraction A of Quillaja Saponaria Molina and fraction C of Quillaja Saponaria Molina in the adjuvant. In embodiments, fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant. In embodiments, fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant. In embodiments, the method comprises administering the immunogenic composition in a prefilled syringe. In embodiments, the dose of antigen administered is from about 0.1 μg to about 100 μg; from about 0.1 μg to about 10 μg; from about 0.1 μg to about 5 μg; from about 0.1 μg to about 3 μg; rom about 0.1 μg to about 2 μg; from about 2 μg to about 10 μg, or from about 2 μg to about 5 μg. In embodiments, the immunogenic composition comprises about 2 μg of antigen. In embodiments, the immunogenic composition comprises about 5 μg of antigen. In embodiments, the immunogenic composition comprises about 10 μg of antigen. In embodiments, the immunogenic composition comprises about 50 μg of antigen. In embodiments, the method comprises administering a first dose and a second dose of the immunogenic composition. In embodiments, the method comprises administering a third dose of the immunogenic composition. In embodiments, the method comprises administering a fourth dose, fifth dose, sixth dose, seventh dose, eighth dose, ninth dose, or tenth dose of the immunogenic composition. In embodiments, the amount of antigen in the second dose is less than the amount of antigen in the first dose. In embodiments, the amount of antigen in the third dose is less than the amount of antigen in the first dose. In embodiments, the first dose and second dose comprise about the same amount of antigen. In embodiments, the second dose and third dose comprise about the same amount of antigen. In embodiments, the first and third dose comprise about the same amount of antigen. In embodiments, the first, second, and third doses comprise about the same amount of antigen. In embodiments, the first, second, third, and fourth doses comprise about the same amount of antigen. In embodiments, the third dose or fourth dose or fifth dose or sixth dose or seventh dose or eighth dose or ninth dose or tenth dose comprises about 90%, about 80%, about 70%, about 60%, about 50%, about 40%, about 30%, about 20%, or about 10% of the antigen in the first dose. In embodiments, the second dose comprises about 90%, about 80%, about 70%, about 60%, about 50%, about 40%, about 30%, about 20%, or about 10% of the antigen in the first dose. In embodiments, the method comprises administering the second dose about 1 month after the first dose. In embodiments, the method comprises administering the second dose about 28 days after the first dose. In embodiments, the method comprises administering the third dose about 56 days after the first dose. In embodiments, the method comprises administering the third dose about 2 months after the first dose. In embodiments, the method comprises administering the third dose about 168 days after the first dose. In embodiments, the method comprises administering the third dose about 6 months after the first dose. In embodiments, the method comprises administering a fourth dose of the immunogenic composition from 12 months to about 16 months after administration of the first dose. In embodiments, the method comprises administering a fourth dose of the immunogenic composition about 15 months after administration of the first dose. In embodiments, the method comprises administering a fourth dose of the immunogenic composition about 421 days after administration of the first dose. In embodiments, the method comprises administering a fourth dose of the immunogenic composition about one year after administration of the third dose. In embodiments, the method comprises administering: (i) a first dose of the immunogenic composition; wherein the first dose comprises about 10 μg of the antigen; (ii) a second dose of the immunogenic composition, wherein the second dose comprises about 10 μg of the antigen; and (iii) a third dose of the immunogenic composition, wherein the third dose comprises about 2 μg of the antigen. In embodiments, the method comprises administering: (i) a first dose of the immunogenic composition; wherein the first dose comprises about 50 μg of the antigen; (ii) a second dose of the immunogenic composition, wherein the second dose comprises about 50 μg of the antigen; and (iii) a third dose of the immunogenic composition, wherein the third dose comprises about 10 μg of the antigen. In embodiments, the method comprises administering: (i) a first dose of the immunogenic composition; wherein the first dose comprises about 10 μg of the antigen; (ii) a second dose of the immunogenic composition, wherein the second dose comprises about 10 μg of the antigen; and (iii) a third dose of the immunogenic composition, wherein the third dose comprises about 10 μg of the antigen. In embodiments, the method comprises administering: (i) a first dose of the immunogenic composition; wherein the first dose comprises about 5 μg of the antigen; (ii) a second dose of the immunogenic composition, wherein the second dose comprises about 5 μg of the antigen, and (iii) a third dose of the immunogenic composition, wherein the third dose comprises about 5 μg of the antigen. In embodiments, the method comprises administering a fourth dose, fifth dose, or both of the immunogenic composition. In embodiments, the method comprises administering the second dose about one month after the first dose and administering the third dose about two months after the second dose. In embodiments, the method comprises administering the second dose about one month after the first dose and administering the third dose about six months after the second dose. In embodiments, the method comprises administering the fourth dose of the immunogenic composition from about 9 months to about two years after administering the first dose of the immunogenic composition. In embodiments, the method comprises administering the fourth dose of the immunogenic composition about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months after administering the first dose of the immunogenic composition. In embodiments, the method comprises administering the fourth dose of the immunogenic composition about ten months after administering the first dose of the immunogenic composition. In embodiments, the method comprises administering the fourth dose of the immunogenic composition about twelve months after administering the first dose of the immunogenic composition. In embodiments, the method comprises administering the fourth dose of the immunogenic composition about fourteen months after administering the first dose of the immunogenic composition. In embodiments, the method comprises administering the fourth dose of the immunogenic composition about fifteen months after administering the first dose of the immunogenic composition. In embodiments, the method comprises administering the fifth dose of the immunogenic composition about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months after administering the fourth dose of the immunogenic composition. In embodiments, the Plasmodium parasite is Plasmodium falciparum, Plasmodium malariae, Plasmodium vivax, Plasmodium ovale, or Plasmodium knowlesi. In embodiments, the CS protein is from Plasmodium falciparum. In embodiments, the method prevents malaria or infection with a Plasmodium parasite with an efficacy from about 50% to about 99%, from about 50% to about 95%, from about 50% to about 90%, from about 50% to about 85%, from about 50% to about 80%, from about 60% to about 99%, from about 65% to about 95%, from about 65% to about 90%, from about 65% to about 85%, from about 69% to about 81%, from about 60% to about 95%, from about 60% to about 90%, from about 60% to about 85%, from about 60% to about 80%, from about 40% to about 99%, from about 40% to about 95%, from about 40% to about 90%, from about 40% to about 85%, from about 40% to about 80%, from about 40% to about 75%, from about 40% to about 70%, from about 40% to about 65%, from about 40% to about 55%, or from about 40% to about 50% for up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, up to about 12 months, up to 13 months, up to 14 months, up to 15 months, up to 16 months, up to 17 months, up to 18 months, up to 19 months, up to 20 months, up to 21 months, up to 22 months, up to 23 months, or up to 24 months after administration of the immunogenic composition. In embodiments, the method prevents malaria or infection with a Plasmodium parasite with an efficacy from about 50% to about 99%, from about 50% to about 95%, from about 50% to about 90%, from about 50% to about 85%, from about 50% to about 80%, from about 60% to about 99%, from about 65% to about 95%, from about 65% to about 90%, from about 65% to about 85%, from about 69% to about 81%, from about 60% to about 95%, from about 60% to about 90%, from about 60% to about 85%, from about 60% to about 80%, from about 40% to about 99%, from about 40% to about 95%, from about 40% to about 90%, from about 40% to about 85%, from about 40% to about 80%, from about 40% to about 75%, from about 40% to about 70%, from about 40% to about 65%, from about 40% to about 55%, or from about 40% to about 50% for at least about 2 months, at least about 2.5 months, at least about 3 months, at least about 3.5 months, at least about 4 months, at least about 4.5 months, at least about 5 months, at least about 5.5 months, at least about 6 months, at least about 6.5 months, at least about 7 months, at least about 7.5 months, at least about 8 months, at least about 8.5 months, at least about 9 months, at least about 9.5 months, at least about 10 months, at least about 10.5 months, at least about 11 months, at least about 11.5 months, at least about 12 months, at least 13 months, at least 14 months, at least 15 months, at least 16 months, at least 17 months, at least 18 months, at least 19 months, at least 20 months, at least 21 months, at least 22 months, at least 23 months, or at least 24 months after administration of the immunogenic composition. In embodiments, the method prevents malaria with an efficacy of about 69% for up to about 12 months. In embodiments, the method prevents malaria with an efficacy of about 69% for at least about 12 months. In embodiments, the method prevents malaria with an efficacy of about 77% for up to about 12 months. In embodiments, the method prevents malaria with an efficacy of about 77% for at least about 12 months. In embodiments, the method prevents malaria with an efficacy of about 77% for up to about 24 months. In embodiments, the method prevents malaria with an efficacy of about 77% for at least about 24 months. In embodiments, the method prevents malaria with an efficacy of about 81% for up to about 12 months. In embodiments, the method prevents malaria with an efficacy of about 81% for at least about 12 months. In embodiments, the method prevents malaria with an efficacy of about 78% for up to about 12 months. In embodiments, the method prevents malaria with an efficacy of about 78% for at least about 12 months.

### BRIEF DESCRIPTION OF THE DRAWINGS

FIG. 1 shows an image of a particle comprising the R21 fusion protein.

FIG. 2 is a graph showing the percentage of subjects over time that are undiagnosed with malaria after the subjects are deliberately infected with human malaria (CHMI) and administered immunogenic compositions of this disclosure. Group 2 was administered an immunogenic composition comprising 10 μg R21 protein and 50 μg of saponin adjuvant (i.e. a saponin adjuvant comprising 85% w/w Fraction A ISCOM matrix and 15% w/w Fraction C ISCOM matrix) at 0 months, 1 months, and 6 months. Group 3 was administered an immunogenic composition comprising 10 μg R21 protein and 50 μg of saponin adjuvant (i.e. a saponin adjuvant comprising 85% w/w Fraction A ISCOM matrix and 15% w/w Fraction C ISCOM matrix) at 0 months, 1 months, and 2 months. Group 6 was a control group that was not administered an immunogenic composition. The efficacy for each group was calculated on day 21 using the equation: 100× (percentage of subjects not administered an immunogenic composition that are diagnosed with malaria-percentage of subjects diagnosed with malaria that are administered an immunogenic composition)/(percentage of subjects not administered an immunogenic composition that are diagnosed with malaria). The efficacy for group 2 was 75%. The efficacy for group 3 was 62.5%. The percentage of controls diagnosed with malaria was 100%.

FIG. 3 is a graph showing the percentage of subjects over time that are undiagnosed with malaria after the subjects are deliberately infected with human malaria (CHMI) Group 2 was administered 10 μg R21 protein and 50 μg of saponin adjuvant (i.e. a saponin adjuvant comprising 85% w/w Fraction A ISCOM matrix and 15% w/w Fraction C ISCOM matrix) at 0 months, 1 months, 6 months, and 14 months. Group 3 was administered 10 μg R21 protein and 50 μg of saponin adjuvant (i.e. a saponin adjuvant comprising 85% w/w Fraction A ISCOM matrix and 15% w/w Fraction C ISCOM matrix) at 0 months, 1, 2, and 10 months. Group 4 was administered 10 μg R21 protein and 50 μg of saponin adjuvant (i.e. a saponin adjuvant comprising 85% w/w Fraction A ISCOM matrix and 15% w/w Fraction C ISCOM matrix) at 0 months, 1 months, and 6 months. Group 7 was a control group that was not administered vaccinations. Efficacy was calculated on day 14 as in FIG. 2. Group 2 exhibited 60% efficacy. Group 3 exhibited 100% efficacy. Group 4 exhibited 40% efficacy. The percentage of controls diagnosed with malaria was 83.3%.

FIG. 4 shows the level of immunoglobulins (as reflected by ELISA units) that protect against malaria in UK adults and Kenya adults. The UK and Kenya adults were administered 10 μg R21 protein and 50 μg of saponin adjuvant (i.e. a saponin adjuvant comprising 85% w/w Fraction A ISCOM matrix and 15% w/w Fraction C ISCOM matrix).

FIG. 5 shows the level of immunoglobulins (as reflected by ELISA units) that protect against malaria in adults, infants, and young children. The adults, infants, and young children were administered 10 μg R21 protein and 50 μg of saponin adjuvant (i.e. a saponin adjuvant comprising 85% w/w Fraction A ISCOM matrix and 15% w/w Fraction C ISCOM matrix).

FIG. 6A shows the dosage regimens of R21 protein and saponin adjuvant (“MM”) evaluated in Example 1.

FIG. 6B shows a graph of the incidence of malaria in patients administered an immunogenic composition comprising 5 μg R21 protein and 25 μg of saponin adjuvant (Group 1), an immunogenic composition comprising 5 μg R21 protein and 50 μg of saponin adjuvant (Group 2), or a control rabies vaccine (i.e. RABIVAX-S). The immunogenic compositions or control rabies vaccine was administered on days 0, 28, and 56. The saponin adjuvant comprised 85% w/w Fraction A ISCOM matrix and 15% w/w Fraction C ISCOM matrix.

FIG. 7A is a graph showing anti-NANP antibody titer in patient sera after administration of one of three immunogenic compositions: (i) an immunogenic composition comprising 5 μg R21 protein and 25 μg of saponin adjuvant (Group 1); (ii) an immunogenic composition comprising 5 μg R21 protein and 50 μg of saponin adjuvant (Group 2); (iii) or a control rabies vaccine (i.e. RABIVAX-S) over time. The saponin adjuvant comprises 85% w/w Fraction A ISCOM matrix and 15% w/w Fraction C ISCOM matrix. The immunogenic compositions or control rabies vaccine was administered on days 0 (“dose 1”), 28 (“dose 2”), 56 (“dose 3”), 421 (“dose 4”). Patients in Group 2 exhibited a higher anti-NANP antibody titer after three doses of vaccine than patients in Group 1 (p<0.0001). The patients in both Group 1 and 2 also exhibited an increase in anti-NANP antibody titer after the boost dose.

FIG. 7B is a graph showing the titer of anti-NANP antibodies in patient sera over time after administration of one of the three immunogenic compositions of FIG. 7A on days 0 (“dose 1”), 28 (“dose 2”), 56 (“dose 3”), 421 (“Boost 1”), and 786 (“Boost 2”).

FIG. 7C is a graph showing the titer of IgG specific for the C-terminus of the CS protein over time in patient sera over time after administration of one of the three immunogenic compositions of FIG. 7A on days 0 (“dose 1”), 28 (“dose 2”), 56 (“dose 3”), 421 (“Boost 1”), and 786 (“Boost 2”).

FIG. 7D is a graph showing the avidity of the CS protein to anti-NANP antibodies from patient sera. The patients were administered an immunogenic compositions comprising 5 μg R21 protein and 25 μg of saponin adjuvant (Group 1) or an immunogenic composition comprising 5 μg R21 protein and 50 μg of saponin adjuvant (Group 2). The immunogenic compositions were administered on days 0, 28, 56, 421, and 786. The avidity was measured 28 days after administration of the dose at day 56 (“D84”), 30 days after administration of the boost dose at day 421 (“1B+30”), and 30 days after administration of the boost dose at day 786 (“2B+30”). The avidity was also measured on day 816 in patients administered the immunogenic compositions on days 0, 28, and 56, but not on days 421 or 786 (“No Boost”).

FIG. 7E is a graph showing the avidity of the CS protein to IgG specific for the C-terminus of the CS protein from patient sera. The patients were administered an immunogenic compositions comprising 5 μg R21 protein and 25 μg of saponin adjuvant (Group 1) or an immunogenic composition comprising 5 μg R21 protein and 50 μg of saponin adjuvant (Group 2). The immunogenic compositions were administered on days 0, 28, 56, 421, and 786. The avidity was measured 28 days after administration of the dose at day 56 (“D84”), 30 days after administration of the boost dose at day 421 (“1B+30”), and 30 days after administration of the boost dose at day 786 (“2B+30”). The avidity was also measured on day 816 in patients administered the immunogenic compositions on days 0, 28, and 56, but not on days 421 or 786 (“No Boost”).

FIG. 8 is a schematic of the primary structure of the R21 protein used in the immunogenic compositions described herein.

FIG. 9A and FIG. 9B show the antibody response induced over time in patients administered different dose regimens of R21 and a saponin adjuvant. FIG. 9A shows the antibody response of groups 1 and 3 (see Table 2 for explanation of regimen). FIG. 9B shows the antibody response of groups 2, 4, and 5 (see Table 2 for explanation of regimen). The saponin adjuvant comprises 85% w/w Fraction A ISCOM matrix and 15% w/w Fraction C ISCOM matrix.

FIG. 10A shows the antibody response (anti-NANP specific IgG) induced by administration of different dosage regimens of Table 2. FIG. 10B shows the antibody response (IgG specific for the C terminus of the CS protein of the R21 protein) induced by administration of different dosage regimens of Table 2 FIG. 10C shows the avidity of the anti-NANP specific IgG of FIG. 10A for the CS protein. FIG. 10D shows the avidity of IgG specific for the C terminus of the CS protein of FIG. 10B for the CS protein.

FIG. 11A shows the correlation of antibody titers from patient sera with protection from malaria. The patients were administered a dose regimen of Group 2 or 3 and were stratified by protection from malaria and unprotection. FIG. 11B shows a correlate of protection in patients of Group 2 and Group 3 with an antibody response EU>1100 or EU<1100. The dose regimens of Group 2 and Group 3 are shown in Table 2.

FIG. 12 shows the dosage regimens of R21 protein and saponin adjuvant (“MM”) evaluated in Example 4.

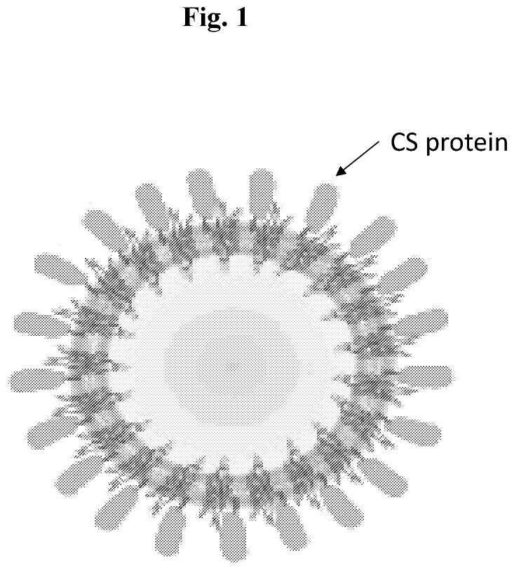

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110.

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen. The immune response may include formation of antibodies and/or a cell-mediated response. Depending on context, the term “vaccine” may also refer to a suspension or solution of an immunogen that is administered to a subject to produce an immune response. Preferably, vaccines induces an immune response that is effective at preventing infection from a Plasmodium parasite.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In some aspects, the adults are seniors about 65 years or older, or about 60 years or older. In some aspects, the subject is a pregnant woman or a woman intending to become pregnant. In other aspects, subject is not a human; for example a non-human primate, for example, a baboon, a chimpanzee, a gorilla, or a macaque. In certain aspects, the subject may be a pet, such as a dog or cat.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

As used herein, the terms “co-formulation mix,” “co-formulation,” “co-formulation vaccine compositions,” “prefilled syringes,” “pre-mix,” refer to vaccine formulations that are prepared for short to long-term storage prior to the time of administration to a subject. Such vaccine formulations contain a combination of antigen and adjuvant in the same container and are prepared in advance of administration. In embodiments, prefilled syringes contain a vaccine formulation comprising the R21 antigen and an adjuvant (e.g., a saponin adjuvant, such as a Matrix adjuvant).

The term “percent identity” in the context of two or more nucleic acid or polypeptide sequences, refers to two or more sequences or subsequences that have a specified percentage of nucleotides or amino acid residues that are the same, when compared. Percentage identity can be calculated using the tools CLUSTALW2, which are available online. The following parameters may be used for CLUSTALW2 Pairwise alignment: Protein Weight Matrix=Gonnet; Gap Open=10; Gap Extension=0.1.

Patients described herein may be diagnosed with primary, secondary, or tertiary clinical malaria. A patient diagnosed with primary clinical malaria exhibits: (a) an axillary temperature ≥37.5° C. and/or history of fever within the last 24 hours and (b) Plasmodium falciparum (P. falciparum) asexual parasitaemia >5000 parasites/μL. A patient diagnosed with secondary clinical malaria exhibits (a) an axillary temperature ≥37.5° C. and/or history of fever within the last 24 hours and (b) P. falciparum asexual parasitaemia >0 parasites/μL or >2500 parasites/μL. A patient diagnosed with tertiary clinical malaria exhibits either (i): (a) an axillary temperature ≥37.5° C. and/or history of fever within the last 24 hours and (b) P. falciparum asexual parasitaemia that is >500 parasites/μL or (ii) (a) an axillary temperature ≥37.5° C. and/or history of fever within the last 24 hours and (b) P. falciparum asexual parasitaemia that is >20,000 parasites/μL or a rapid detection test is positive for malaria antigens.

The term “efficacy” (also referred to herein as “vaccine efficacy”) of an immunogenic composition described herein refers to the percentage reduction of disease (e.g., malaria) in a group administered an immunogenic composition as compared to a group that is not administered the immunogenic composition. In embodiments, efficacy (E) is calculated using the following equation: 100×(percentage of subjects not administered an immunogenic composition that are diagnosed with disease-percentage of subjects diagnosed with malaria that are administered an immunogenic composition)/(percentage of subjects not administered an immunogenic composition that are diagnosed with disease).

### Immunogenic Compositions Containing Circumsporozoite (CS) Proteins, Hepatitis B Surface Antigen, Saponin Adjuvants, and Combinations Thereof

Disclosed herein are immunogenic compositions comprising an antigen of a Plasmodium parasite. In embodiments, the Plasmodium parasite is Plasmodium falciparum, Plasmodium malariae, Plasmodium vivax, Plasmodium ovale, or Plasmodium knowlesi. In embodiments, the antigen comprises a circumsporozoite (CS) protein or fragment thereof.

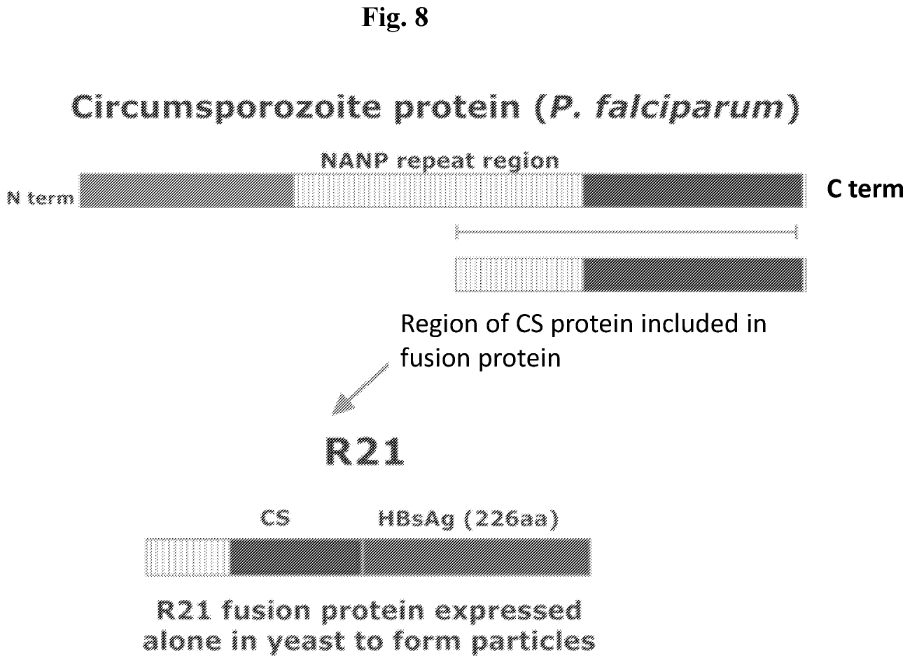

A CS protein is comprised of an N-terminus that binds heparin sulfate proteoglycans, a four amino acid repeat region (NANP), and a C terminus that contains a thrombospondin-like type I repeat (TSR) domain. In embodiments, the antigen comprises the N-terminus, NANP, TSR domain, or a combination thereof of a CS protein. In embodiments, the antigen comprises the C terminus of TSR or a fragment thereof and the NANP or a fragment thereof (e.g., FIG. 8).

In embodiments, the antigen comprises a hepatitis B surface antigen (HBsAg) or fragment thereof. In embodiments, the HBsAg fragment is about 226 amino acids. In embodiments, the HBsAg fragment comprises from about 180 amino acids to about 220 amino acids.

In embodiments, the antigen is a fusion protein comprising a CS protein or fragment thereof and a HBsAg or fragment thereof. In embodiments, the antigen has the amino acid sequence of SEQ ID NO: 1:

(SEQ ID NO: 1)
MDPNANPNANPNANPNANPNANPNANPNANPNANPNANPNANPNA
NPNANPNANPNANPNANPNANPNANPNANPNKNNQGNGQGHNMPN
DPNRNVDENANANSAVKNNNNEEPSDKHIKEYLNKIQNSLSTEWS
PCSVTCGNGIQVRIKPGSANKPKDELDYANDIEKKICKMEKCSSV
PVTNMENITSGFLGPLLVLQAGFFLLTRILTIPQSLDSWWTSLNF
LGGSPVCLGQNSQSPTSNHSPTSCPPICPGYRWMCLRRFIIFLFI
LLLCLIFLLVLLDYQGMLPVCPLIPGSTTTNTGPCKTCTTPAQGN
SMFPSCCCTKPTDGNCTCIPIPSSWAFAKYLWEWASVRFSWLSLL
VPFVQWFVGLSPTVWLSAIWMMWYWGPSLYSIVSPFIPLLPIFFC
LWVYI

The antigen with the amino acid sequence of SEQ ID NO: 1 comprises a C-terminus of the CS protein (underlined, SEQ ID NO: 8), a NANP repeat region (bolded, SEQ ID NO: 7), and a HBsAg antigen (italicized, SEQ ID NO: 9). In embodiments, the antigen comprises a NANP repeat region having at least 80%, at least 85%, at least 90%, at least 91%, at least 92%, at least 93%, at least 94%, at least 95%, at least 96%, at least 97%, at least 98%, at least 99%, or 100% identity to a polypeptide having the amino acid sequence of SEQ ID NO: 7. In embodiments, the antigen comprises a C-terminus of the CS protein having at least 80%, at least 85%, at least 90%, at least 91%, at least 92%, at least 93%, at least 94%, at least 95%, at least 96%, at least 97%, at least 98%, at least 99%, or 100% identity to a polypeptide having the amino acid sequence of SEQ ID NO: 8. In embodiments, the antigen comprises a HBsAg having at least 80%, at least 85%, at least 90%, at least 91%, at least 92%, at least 93%, at least 94%, at least 95%, at least 96%, at least 97%, at least 98%, at least 99%, or 100% identity to a polypeptide having the amino acid sequence of SEQ ID NO: 9.

In embodiments, the antigen comprises a fragment thereof of a polypeptide of SEQ ID NO: 1.

In embodiments, the amino acid sequence of the antigen has at least 80%, at least 85%, at least 90%, at least 91%, at least 92%, at least 93%, at least 94%, at least 95%, at least 96%, at least 97%, at least 98%, at least 99%, or 100% identity to a protein having the amino acid sequence of SEQ ID NO: 1. In embodiments, the protein having an amino acid sequence of SEQ ID NO: 1 is referred to herein as “R21.” Alternative fusion proteins that may be utilized in the immunogenic compositions herein are described in U.S. Publication No. 2020/0,207,811, which is incorporated by reference herein in its entirety for all purposes.

In embodiments, a “fragment thereof” of a protein is between 10 and 1500 amino acids in length (e.g. about 10, about 20, about 30, about 40, about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, or about 1500 amino acids in length). In embodiments, a fragment thereof comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 97% of the amino acids of the wild-type version of the protein

In embodiments, the antigen has the amino acid sequence of SEQ ID NO: 2:

MDPNANPNANPNANPNANPNANPNANPNANPNANPNANPNANPNA
NPNANPNANPNANPNANPNANPNANPNANPNKNNQGNGQGHNMPN
DPNRNVDENANANSAVKNNNNEEPSDKHIKEYLNKIQNSLSTEWS
PCSVTCGNGIQVRIKPGSANKPKDELDYANDIEKKICKMEKCSSV
PVTNMENITSGFLGPLLVLQAGFFLLTRILTIPQSLDSWWTSLNF
LGGSPVCLGQNSQSPTSNHSPTSCPPICPGYRWMCLRRFIIFLFI
LLLCLIFLLVLLDYQGMLPVCPLIPGSTTTNTGPCKTCTTPAQGN
SMFPSCCCTKPTDGNCTCIPIPSSWAFAKYLWEWASVRFSWLSLL
VPFVQWFVGLSPTVWLSAIWMMWYWGPSLYSIVSPFIPLLPIFFC
LWVYIEPEA

In embodiments, the amino acid sequence of the antigen has at least 80%, at least 85%, at least 90%, at least 91%, at least 92%, at least 93%, at least 94%, at least 95%, at least 96%, at least 97%, at least 98%, at least 99%, or 100% identity to a protein having the amino acid sequence of SEQ ID NO: 2.

In embodiments, the nucleic acid encoding the antigen of the Plasmodium parasite is extended at the 5′end, 3′ end, or both. In embodiments, the extension encodes a protein tag used for purification or detection. In embodiments, the protein tag is a polyglutamate tag, a FLAG-tag, a HA-tag, a polyHis-tag (having about 5-10 histidines) (SEQ ID NO: 3), a hexahistidine tag (SEQ ID NO: 4), an 8×-His-tag (having eight histidines) (SEQ ID NO: 5), a Myc-tag, a Glutathione-S-transferase-tag, a Green fluorescent protein-tag, Maltose binding protein-tag, a Thioredoxin-tag, an Fc-tag, or a C-tag. In embodiments, the extension comprises a C-tag. A C-tag comprises the sequence EPEA (SEQ ID NO: 6).

In embodiments, the nucleic acid encoding the antigen is cloned into a Hansenula polymorpha (H. polymorpha) expression plasmid. In embodiments, the H. polymorpha plasmid is pFPMT121. In embodiments, the pFPMT121 plasmid carries the LEU2 gene for selection in yeast. In embodiments, the auxotrophic H. polymorpha yeast strain ALU3 (relevant genotype: ade1, leu2, ura3) is used as an expression host. In embodiments, yeast cells carrying the plasmid comprising the antigen are produced by electroporation. In embodiments, gene expression of the antigen is induced by introducing methanol (e.g., 1% v/v) to the yeast cells. In embodiments, antigen is isolated by ultracentrifugation.

In embodiments, the antigen is expressed in Pichia pastoris cells. In embodiments, after expression in P. pastoris cells, the yeast cell membranes are lysed. In embodiments, the cells are lysed with a lysis reagent selected from Tris buffer (e.g., 10 mM, pH 7.8), Triton X-100 (e.g., 0.1%), EDTA (e.g., 1 mM), benzonase (e.g., 250 U/mL), or a combination thereof. In embodiments, a mixture comprising lysis reagents and yeast cells is vortexed. In embodiments, cell debris is removed from lysed cells by centrifugation. In embodiments, the yeast lysate is layered onto a discontinuous CsCl gradient and ultracentrifuged. In embodiments, the antigen is purified using. In embodiments, the antigen is eluted in Tris buffer (e.g., 10 mM). In embodiments, the antigen is desalted.

In embodiments, the antigen is purified via affinity chromatography. In embodiments, purification comprises exposing an antigen comprising a C-tag (e.g., the antigen of SEQ ID NO: 2) to a C-tag affinity chromatography column; removing impurities using a buffer containing 20 mM Tris and 0.1 M MgCl₂, wherein the pH of the buffer is about 7; and eluting the antigen in a buffer comprising 20 mM Tris and 2 M MgCl₂, wherein the pH of the buffer is about 7.

The following articles describe methods of expressing and purifying the antigens of the disclosure: Kurtovic et al. Front Immunol. 2021; 12:641421; Degelmann et al. FEMS Yeast Rest (2002) 2 (3): 349-361; Lahtchev et al. Arch Microbiol (2002) 177 (2): 150-158; Faber et al. Curr Genet (1994) 25 (4): 305-310; Guengerich et al. Genet Biotechnol: Springer (2004) 2:273-87; Mukhopadhyay et al. Biotechnol Bioeng. (2022) 119 (10): 2784-2793; Datoo et al. Lancer. (2021); 397:1809-1818; and Datoo et al. Lancet Infect Dis (Sep. 7, 2022), doi.org/10.1016/S1473-3099 (22) 00442-X. Each of the aforementioned references is incorporated by reference herein in its entirety for all purposes.

In embodiments, the antigen is produced in yeast. In embodiments, the yeast is selected from the group consisting of: Saccharomyces cerevisiae, Pichia pastoris, Hansemda polymorpha (also referred to as “Ogataea polymorpha”), Schizosaccharomyces pombe, Kluyveromyces lactis, Kluyveromyces thermotolerans, Candida glabrata, Candida albicans, Pichia stipites, Yarrowia lipolytica, Ogataea minuta, Candida boidinii, and Kluyveromyces lactis.

### Adjuvants

In embodiments, the immunogenic compositions comprise an adjuvant.

In certain embodiments, the compositions disclosed herein may be combined with one or more adjuvants to enhance an immune response. In other embodiments, the compositions are prepared without adjuvants, and are thus available to be administered as adjuvant-free compositions. Advantageously, adjuvant-free compositions disclosed herein may provide protective immune responses when administered as a single dose. Alum-free compositions that induce robust immune responses are especially useful in adults about 60 and older.

### Aluminum-Based Adjuvants

In embodiments, the adjuvant may be alum (e.g. AlPO₄ or Al(OH)₃). Typically, the nanoparticle is substantially bound to the alum. For example, the nanoparticle may be at least 80% bound, at least 85% bound, at least 90% bound or at least 95% bound to the alum. Often, the nanoparticle is 92% to 97% bound to the alum in a composition. The amount of alum is present per dose is typically in a range between about 400 μg to about 1250 μg. For example, the alum may be present in a per dose amount of about 300 μg to about 900 μg, about 400 μg to about 800 μg, about 500 μg to about 700 μg, about 400 μg to about 600 μg, or about 400 μg to about 500 μg. Typically, the alum is present at about 400 μg for a dose of 120 μg of the protein nanoparticle.

In embodiments, the adjuvant is a saponin adjuvant.

### Saponin Adjuvants

Adjuvants containing saponin may also be combined with the immunogens disclosed herein. Saponins are glycosides derived from the bark of the Quillaja saponaria Molina tree. Typically, saponin is prepared using a multi-step purification process resulting in multiple fractions. As used, herein, the term “a saponin fraction from Quillaja saponaria Molina” is used generically to describe a semi-purified or defined saponin fraction of Quillaja saponaria or a substantially pure fraction thereof.

### Saponin Fractions

Several approaches for producing saponin fractions are suitable. Fractions A, B, and C are described in U.S. Pat. No. 6,352,697 and may be prepared as follows. A lipophilic fraction from Quil A, a crude aqueous Quillaja saponaria Molina extract, is separated by chromatography and eluted with 70% acetonitrile in water to recover the lipophilic fraction. This lipophilic fraction is then separated by semi-preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluted at approximately 49% acetonitrile. Additional information regarding purification of Fractions is found in U.S. Pat. No. 5,057,540. When prepared as described herein, Fractions A, B and C of Quillaja saponaria Molina each represent groups or families of chemically closely related molecules with definable properties. The chromatographic conditions under which they are obtained are such that the batch-to-batch reproducibility in terms of elution profile and biological activity is highly consistent.

Other saponin fractions have been described. Fractions B3, B4 and B4b are described in EP 0436620. Fractions QA1-QA22 are described EP03632279 B2, Q-VAC (Nor-Feed, AS Denmark), Quillaja saponaria Molina Spikoside (Isconova AB, Ultunaallén 2B, 756 51 Uppsala, Sweden). Fractions QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20, QA-21, and QA-22 of EP 0 3632 279 B2, especially QA-7, QA-17, QA-18, and QA-21 may be used. They are obtained as described in EP 0 3632 279 B2, especially at page 6 and in Example 1 on page 8 and 9.

The saponin fractions described herein and used for forming adjuvants are often substantially pure fractions; that is, the fractions are substantially free of the presence of contamination from other materials. In particular aspects, a substantially pure saponin fraction may contain up to 40% by weight, up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight, or up to 0.1% by weight of other compounds such as other saponins or other adjuvant materials.

### ISCOM Structures

Saponin fractions may be administered in the form of a cage-like particle referred to as an ISCOM (Immune Stimulating COMplex). ISCOMs may be prepared as described in EP0109942B1, EP0242380B1 and EP0180546 B1. In particular embodiments a transport and/or a passenger antigen may be used, as described in EP 9600647-3 (PCT/SE97/00289).

### Matrix Adjuvants

In embodiments, the ISCOM is an ISCOM matrix complex. An ISCOM matrix complex comprises at least one saponin fraction and a lipid. The lipid is at least a sterol, such as cholesterol. In particular aspects, the ISCOM matrix complex also contains a phospholipid. The ISCOM matrix complexes may also contain one or more other immunomodulatory (adjuvant-active) substances, not necessarily a glycoside, and may be produced as described in EP0436620B1, which is incorporated by reference in its entirety herein.

In other aspects, the ISCOM is an ISCOM complex. An ISCOM complex contains at least one saponin, at least one lipid, and at least one kind of antigen or epitope. The ISCOM complex contains antigen associated by detergent treatment such that that a portion of the antigen integrates into the particle. In contrast, ISCOM matrix is formulated as an admixture with antigen and the association between ISCOM matrix particles and antigen is mediated by electrostatic and/or hydrophobic interactions.

According to one embodiment, the saponin fraction integrated into an ISCOM matrix complex or an ISCOM complex, or at least one additional adjuvant, which also is integrated into the ISCOM or ISCOM matrix complex or mixed therewith, is selected from fraction A, fraction B, or fraction C of Quillaja saponaria, a semipurified preparation of Quillaja saponaria, a purified preparation of Quillaja saponaria, or any purified sub-fraction e.g., QA 1-21.

In particular aspects, each ISCOM particle may contain at least two saponin fractions. Any combinations of weight % of different saponin fractions may be used. Any combination of weight % of any two fractions may be used. For example, the particle may contain any weight % of fraction A and any weight % of another saponin fraction, such as a crude saponin fraction or fraction C, respectively. Accordingly, in particular aspects, each ISCOM matrix particle or each ISCOM complex particle may contain from 0.1 to 99.9 by weight, S to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. fraction A and the rest up to 100% in each case of another saponin e.g. any crude fraction or any other faction e.g. fraction C. The weight is calculated as the total weight of the saponin fractions. Examples of ISCOM matrix complex and ISCOM complex adjuvants are disclosed in U.S. Published Application No. 2013/0129770, which is incorporated by reference in its entirety herein.

In particular embodiments, the ISCOM matrix or ISCOM complex comprises from 5-99% by weight of one fraction, e.g., fraction A and the rest up to 100% of weight of another fraction e.g. a crude saponin fraction or fraction C. The weight is calculated as the total weight of the saponin fractions.

In another embodiment, the ISCOM matrix or ISCOM complex comprises from 40% to 99% by weight of one fraction, e.g. fraction A and from 1% to 60% by weight of another fraction, e.g. a crude saponin fraction or fraction C. The weight is calculated as the total weight of the saponin fractions.

In yet another embodiment, the ISCOM matrix or ISCOM complex comprises from 70% to 95% by weight of one fraction e.g., fraction A, and from 30% to 5% by weight of another fraction, e.g., a crude saponin fraction, or fraction C. The weight is calculated as the total weight of the saponin fractions. In other embodiments, the saponin fraction from Quillaja saponaria Molina is selected from any one of QA 1-21.

In addition to particles containing mixtures of saponin fractions, ISCOM matrix particles and ISCOM complex particles may each be formed using only one saponin fraction. Compositions disclosed herein may contain multiple particles wherein each particle contains only one saponin fraction. That is, certain compositions may contain one or more different types of ISCOM-matrix complexes particles and/or one or more different types of ISCOM complexes particles, where each individual particle contains one saponin fraction from Quillaja saponaria Molina, wherein the saponin fraction in one complex is different from the saponin fraction in the other complex particles.

In particular aspects, one type of saponin fraction or a crude saponin fraction may be integrated into one ISCOM matrix complex or particle and another type of substantially pure saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which one saponin fraction Quillaja saponaria Molina and another saponin fraction Quillaja saponaria Molina are separately incorporated into different ISCOM matrix complex particles and/or ISCOM complex particles.

The ISCOM matrix or ISCOM complex particles, which each have one saponin fraction, may be present in composition at any combination of weight %. In particular aspects, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction. In some aspects, the remaining portion is one or more ISCOM matrix or complexes where each matrix or complex particle contains only one saponin fraction. In other aspects, the ISCOM matrix or complex particles may contain more than one saponin fraction.

In particular compositions, the only saponin fraction in a first ISCOM matrix or ISCOM complex particle is Fraction A and the only saponin fraction in a second ISCOM matrix or ISCOM complex particle is Fraction C.

Preferred compositions comprise a first ISCOM matrix containing Fraction A and a second ISCOM matrix containing Fraction C, wherein the Fraction A ISCOM matrix constitutes about 70% per weight of the total saponin adjuvant, and the Fraction C ISCOM matrix constitutes about 30% per weight of the total saponin adjuvant. In another preferred composition, the Fraction A ISCOM matrix constitutes about 85% per weight of the total saponin adjuvant, and the Fraction C ISCOM matrix constitutes about 15% per weight of the total saponin adjuvant. Thus, in certain compositions, the Fraction A ISCOM matrix is present in a range of about 70% to about 85%, and Fraction C ISCOM matrix is present in a range of about 15% to about 30%, of the total weight amount of saponin adjuvant in the composition. In embodiments, the Fraction A ISCOM matrix accounts for 50-96% by weight and Fraction C ISCOM matrix accounts for the remainder, respectively, of the sums of the weights of Fraction A ISCOM matrix and Fraction C ISCOM in the adjuvant. In embodiments, the Fraction A ISCOM matrix accounts for at least 75% by weight and Fraction C ISCOM matrix accounts for the remainder, respectively, of the sums of the weights of Fraction A ISCOM matrix and Fraction C ISCOM in the adjuvant. In embodiments, the Fraction A ISCOM matrix is present at about 92% and Fraction C ISCOM matrix is present at about 8% of the total weight amount of saponin adjuvant in the composition. In a particularly preferred composition, referred to herein as MATRIX-M™, the Fraction A ISCOM matrix is present at about 85% and Fraction C ISCOM matrix is present at about 15% of the total weight amount of saponin adjuvant in the composition. MATRIX-M™ may be referred to interchangeably as Matrix-M1.

Exemplary QS-7 and QS-21 fractions, their production and their use is described in U.S. Pat. Nos. 5,057,540; 6,231,859; 6,352,697, 6,524,584; 6,846,489; 7,776,343, and 8,173,141, which are incorporated by reference herein.

In some, compositions other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/TWEEN® polysorbate 80 emulsion. In embodiments, the adjuvant may be a paucilamellar lipid vesicle; for example, NOVASOMES®. NOVASOMES® are paucilamellar nonphospholipid vesicles ranging from about 100 nm to about 500 nm. They comprise BRIJ® alcohol ethoxylate 72, cholesterol, oleic acid and squalene. NOVASOMES® have been shown to be an effective adjuvant (see, U.S. Pat. Nos. 5,629,021, 6,387,373, and 4,911,928.

### Excipients

In embodiments, the immunogenic compositions described herein comprise various excipients, buffers, and the like. For example, the immunogenic compositions may contain sodium phosphate, sodium chloride, and/or histidine. Sodium phosphate may be present at about 10 mM to about 50 mM, about 15 mM to about 25 mM, or about 25 mM; in particular cases, about 22 mM sodium phosphate is present. Histidine may be present about 0.1% (w/v), about 0.5% (w/v), about 0.7% (w/v), about 1% (w/v), about 1.5% (w/v), about 2% (w/v), or about 2.5% (w/v). Sodium chloride, when present, may be about 150 mM. In certain compositions, the sodium chloride may be present in higher concentrations, for example from about 200 mM to about 500 mM. In embodiments, the sodium chloride is present in a high concentration, including but not limited to about 200 mM, about 250 mM, about 300 mM, about 350 mM, about 400 mM, about 450 mM, or about 500 mM.

### Administration and Dosage

Provided herein are novel methods for administering the aforementioned immunogenic compositions. In embodiments, the immunogenic compositions induce an immune response against malaria in a subject in need thereof. In embodiments, the immunogenic compositions induce an immune response against a parasite selected from Plasmodium falciparum, Plasmodium malariae, Plasmodium vivax, Plasmodium ovale, or Plasmodium knowlesi.

In embodiments, the immunogenic compositions described herein are administered in a single dose. In embodiments, the immunogenic compositions described herein are administered in multiple doses. For example, in embodiments, one, two, three, four, five, six, seven, eight, nine, or ten doses of the immunogenic composition is administered. These doses are referred to as a first, second, third, fourth, fifth, sixth, seventh, eight, ninth, or tenth dose.

In embodiments, the amount of antigen that is administered per dose ranges from about 0.1 μg to about 100 μg, including all ranges and subranges therebetween. For example, about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg of antigen may be administered per dose of immunogenic composition. In embodiments, the antigen is R21 or an antigen with at least 70%, at least 80%, at least 90%, at least 91%, at least 92%, at least 93%, at least 94%, at least 95%, at least 96%, at least 97%, at least 98%, at least 99%, or 100% identity to the polypeptide of SEQ ID NO: 1. In embodiments, the antigen has at least 70%, at least 80%, at least 90%, at least 91%, at least 92%, at least 93%, at least 94%, at least 95%, at least 96%, at least 97%, at least 98%, at least 99%, or 100% identity to the polypeptide of SEQ ID NO: 2.

In embodiments, the immunogenic compositions comprise an adjuvant. In embodiments, the amount of adjuvant in each dose of immunogenic composition ranges from about 1 μg to about 100 μg. For example, in embodiments, the amount of adjuvant in each dose of immunogenic composition is about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg. In embodiments, the adjuvant is a saponin adjuvant. In embodiments, the saponin adjuvant comprises 85% w/w Fraction A ISCOM matrix and 15% w/w Fraction C ISCOM matrix. In embodiments, the saponin adjuvant comprises about 25 μg of 85% w/w Fraction A ISCOM matrix and 15% w/w Fraction C ISCOM matrix. In embodiments, the saponin adjuvant comprises about 50 μg of 85% w/w Fraction A ISCOM matrix and 15% w/w Fraction C ISCOM matrix.

In embodiments, the second, third, fourth, fifth, sixth, seventh, eight, ninth, or tenth dose of an immunogenic composition is administered about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, or about 52 weeks after administration of the first dose. In embodiments, the second, third, fourth, fifth, sixth, seventh, eight, ninth, or tenth dose of an immunogenic composition is administered about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, about 90 days, about 91 days, about 92 days, about 93 days, about 94 days, about 95 days, about 96 days, about 97 days, about 98 days, about 99 days, about 100 days, about 101 days, about 102 days, about 103 days, about 104 days, about 105 days, about 106 days, about 107 days, about 108 days, about 109 days, about 110 days, about 111 days, about 112 days, about 113 days, about 114 days, about 115 days, about 116 days, about 117 days, about 118 days, about 119 days, about 120 days, about 121 days, about 122 days, about 123 days, about 114 days, about 115 days, about 116 days, about 117 days, about 118 days, about 119 days, about 120 days, about 121 days, about 122 days, about 123 days, about 124 days, about 125 days, about 126 days, about 127 days, about 128 days, about 129 days, about 130 days, about 131 days, about 132 days, about 133 days, about 134 days, about 135 days, about 136 days, about 137 days, about 138 days, about 139 days, about 140 days, about 141 days, about 142 days, about 143 days, about 144 days, about 145 days, about 146 days, about 147 days, about 148 days, about 149 days, about 150 days, about 151 days, about 152 days, about 153 days, about 154 days, about 155 days, about 156 days, about 157 days, about 158 days, about 159 days, about 160 days, about 161 days, about 162 days, about 163 days, about 164 days, about 165 days, about 166 days, about 167 days, about 168 days, about 169 days, about 170 days, about 171 days, about 172 days, about 173 days, about 174 days, about 175 days, about 176 days, about 177 days, about 178 days, about 179 days, about 180 days, about 181 days, about 182 days, about 183 days, about 184 days, about 185 days, about 186 days, about 187 days, about 188 days, about 189 days, about 190 days, about 191 days, about 192 days, about 193 days, about 194 days, about 195 days, about 196 days, about 197 days, about 198 days, about 199 days, about 200 days, about 201 days, about 202 days, about 203 days, about 204 days, about 205 days, about 206 days, about 207 days, about 208 days, about 209 days, about 210 days, about 211 days, about 212 days, about 213 days, about 214 days, about 215 days, about 216 days, about 217 days, about 218 days, about 219 days, about 220 days, about 221 days, about 222 days, about 223 days, about 224 days, about 225 days, about 226 days, about 227 days, about 228 days, about 229 days, about 230 days, about 231 days, about 232 days, about 233 days, about 234 days, about 235 days, about 236 days, about 237 days, about 238 days, about 239 days, about 240 days, about 241 days, about 242 days, about 243 days, about 244 days, about 245 days, about 246 days, about 247 days, about 248 days, about 249 days, about 250 days, about 251 days, about 252 days, about 253 days, about 254 days, about 255 days, about 256 days, about 257 days, about 258 days, about 259 days, about 260 days, about 261 days, about 262 days, about 263 days, about 264 days, about 265 days, about 266 days, about 267 days, about 268 days, about 269 days, about 270 days, about 271 days, about 272 days, about 273 days, about 274 days, about 275 days, about 276 days, about 277 days, about 278 days, about 279 days, about 280 days, about 281 days, about 282 days, about 283 days, about 284 days, about 285 days, about 286 days, about 287 days, about 288 days, about 289 days, about 290 days, about 291 days, about 292 days, about 293 days, about 294 days, about 295 days, about 296 days, about 297 days, about 298 days, about 299 days, about 300 days, about 301 days, about 302 days, about 303 days, about 304 days, about 305 days, about 306 days, about 307 days, about 308 days, about 309 days, about 310 days, about 311 days, about 312 days, about 313 days, about 314 days, about 315 days, about 316 days, about 317 days, about 318 days, about 319 days, about 320 days, about 321 days, about 322 days, about 323 days, about 324 days, about 325 days, about 326 days, about 327 days, about 328 days, about 329 days, about 330 days, about 331 days, about 332 days, about 333 days, about 334 days, about 335 days, about 336 days, about 337 days, about 338 days, about 339 days, about 340 days, about 341 days, about 342 days, about 343 days, about 344 days, about 345 days, about 346 days, about 347 days, about 348 days, about 349 days, about 350 days, about 351 days, about 352 days, about 353 days, about 354 days, about 355 days, about 356 days, about 357 days, about 358 days, about 359 days, about 360 days, about 361 days, about 362 days, about 363 days, about 364 days, about 365 days, about 13 months, about 14 months, about 15 months, about 16 months, about 17 months, about 18 months, about 19 months, about 20 months, about 21 months, about 22 months, about 23 months, about 24 months, about 2.5 years, about 3 years, about 3.5 years, about 4 years, about 4.5 years, about 5 years, about 5.5 years, about 6 years, about 6.5 years, about 7 years, about 8 years, about 9 years, about 10 years, about 11 years, about 12 years, about 13 years, about 14 years, about 15 years, about 16 years, about 17 years, about 18 years, about 19 years, or about 20 years after administration of the first dose. In embodiments, the second dose is administered about 28 days after administration of the first dose. In embodiments, the third dose is administered about 56 days after administration of the first dose. In embodiments, the third dose is administered about 168 days after administration of the first dose. In embodiments, a second dose is administered about 28 days after administration of the first dose and a third dose is administered about 56 days after the first dose. In embodiments, a second dose is administered about 28 days after administration of the first dose and a third dose is administered about 168 days after the first dose.

In embodiments, the second dose of the immunogenic composition is administered about 1 month (e.g., 28 days, 29 days, 30 days, or 31 days) after the first dose of immunogenic composition. In embodiments, the third dose of immunogenic composition is administered from about 2 months to about 24 months, from about 2 months to about 6 months, from about 10 months to about 15 months, from about 12 months to about 18 months, or from about 12 months to about 16 months after the first dose of immunogenic composition. In embodiments, the fourth dose of immunogenic composition is administered from about 2 months to about 24 months, from about 2 months to about 6 months, from about 10 months to about 15 months, from about 12 months to about 18 months, or from about 12 months to about 16 months after the first dose of immunogenic composition. In embodiments, the third dose of immunogenic composition is administered at least 2 months, at least 3 months, at least 4 months, at least 5 months, at least 6 months, at least 7 months, at least 8 months, at least 9 months, at least 10 months, at least 11 months, at least 1 year, at least 13 months, at least 14 months, or at least 15 months after the first dose of immunogenic composition is administered. In embodiments, the third dose of immunogenic composition is administered up to 2 months, up to 3 months, up to 4 months, up to 5 months, up to 6 months, up to 7 months, up to 8 months, up to 9 months, up to 10 months, up to 11 months, up to 1 year, up to 13 months, up to 14 months, or up to 15 months after the first dose of immunogenic composition is administered. In embodiments, the fourth dose of immunogenic composition is administered at least 2 months, at least 3 months, at least 4 months, at least 5 months, at least 6 months, at least 7 months, at least 8 months, at least 9 months, at least 10 months, at least 11 months, at least 1 year, at least 13 months, at least 14 months, or at least 15 months after the first dose of immunogenic composition is administered. In embodiments, the fourth dose of immunogenic composition is administered up to 2 months, up to 3 months, up to 4 months, up to 5 months, up to 6 months, up to 7 months, up to 8 months, up to 9 months, up to 10 months, up to 11 months, up to 1 year, up to 13 months, up to 14 months, up to 15 months, up to 16 months, up to 17 months, up to 18 months, up to 19 months, up to 20 months, up to 21 months, up to 22 months, up to 23 months, or up to 24 months after the first dose of immunogenic composition is administered.

In embodiments, when a dose subsequent to the first dose is administered, the subsequent dose may comprise an amount of of antigen that is less than the amount of antigen in the first dose. For example, if the first dose comprises 5 μg of antigen, the third dose may comprise 1 μg of antigen. In embodiments, the subsequent dose is about 1%, about 2%, about 3% about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% lower than the first dose. For example, if the first dose comprises 5 μg antigen, a subsequent dose that is 50% lower than the first dose comprises 2.5 μg antigen. In embodiments, the third dose contains one fifth the antigen of the first dose. In embodiments, the first dose comprises 10 μg of antigen and the third dose comprises 2 μg of antigen. In embodiments, the first dose comprises 50 μg of antigen and the third dose comprises 10 μg of antigen.

In embodiments, three doses of an immunogenic composition described herein are administered, a first dose, a second dose, and a third dose. In embodiments, the first dose comprises 10 μg of antigen, the second dose comprises 10 μg of antigen, and the third dose comprises 10 μg of antigen. In embodiments, the first dose comprises 5 μg of antigen, the second dose comprises 5 μg of antigen, and the third dose comprises 5 μg of antigen. In embodiments, the first dose comprises 50 μg of antigen, the second dose comprises 50 μg of antigen, and the third dose comprises 10 μg of antigen. In embodiments, the first dose comprises 10 μg of antigen, the second dose comprises 10 μg of antigen, and the third dose comprises 2 μg of antigen.

In embodiments, four doses of an immunogenic composition described herein are administered, a first dose, a second dose, third dose, and fourth dose. In embodiments, the first dose comprises 10 μg of antigen, the second dose comprises 10 μg of antigen, the third dose comprises 10 μg of antigen, and the fourth dose comprises 10 μg of antigen. In embodiments, the first dose comprises 5 μg of antigen, the second dose comprises 5 μg of antigen, the third dose comprises 5 μg of antigen, and the fourth dose comprises 5 μg of antigen.

In embodiments, five doses of an immunogenic composition described herein are administered, a first dose, a second dose, third dose, fourth dose, and a fifth dose. In embodiments, the first dose comprises 10 μg of antigen, the second dose comprises 10 μg of antigen, the third dose comprises 10 μg of antigen, the fourth dose comprises 10 μg of antigen, and the fifth dose comprises 10 μg of antigen. In embodiments, the first dose comprises 5 μg of antigen, the second dose comprises 5 μg of antigen, the third dose comprises 5 μg of antigen, the fourth dose comprises 5 μg of antigen, and the fifth dose comprises 5 μg of antigen.

In embodiments, the method comprises administering an immunogenic composition according to a dose regiment of Table A, wherein the amount refers to the dose of antigen and the days or months of the second, third, fourth, or fifth dose are days or months after the first dose.

TABLE A
Dosage Regimens

| | First | Second | Third | Fourth | Fifth |
|---|---|---|---|---|---|
| Regimen | Dose | Dose | Dose | Dose | Dose |
| 1 | 10 μg | 10 μg (28 | 10 μg (56 | — | |
| | | days) | days) | | |
| 2 | 10 μg | 10 μg (28 | 10 μg (168 | — | |
| | | days) | days) | | |
| 3 | 50 μg | 50 μg (28 | 10 μg (168 | — | |
| | | days) | days) | | |
| 4 | 10 μg | 10 μg (28 | 2 μg (168 | — | |
| | | days) | days) | | |
| 5 | 10 μg | 10 μg (1 | 10 μg (2 | — | |
| | | month) | months) | | |
| 6 | 10 μg | 10 μg (1 | 10 μg (6 | — | |
| | | month) | months) | | |
| 7 | 50 μg | 50 μg (1 | 10 μg (6 | — | |
| | | month) | months) | | |
| 8 | 10 μg | 10 μg (1 | 2 μg (6 | — | |
| | | month) | months) | | |
| 9 | 10 μg | 10 μg (1 | 10 μg (2 | 10 μg (14 | |
| | | month) | months) | months) | |
| 10 | 5 μg | 5 μg (1 | 5 μg (2 | 5 μg (15 | |
| | | month) | months) | months) | |
| 11 | 10 μg | 10 μg (1 | 10 μg (6 | 10 μg (14 | |
| | | month) | months) | months) | |
| 12 | 10 μg | 10 μg (1 | 10 μg (2 | 10 μg (10 | |
| | | month) | months) | months) | |
| 13 | 10 μg | 10 μg (1 | 10 μg (6 | | |
| | | month) | months) | | |
| 14 | 10 μg | 10 μg (1 | 10 μg (2 | | |
| | | month) | months) | | |
| 15 | 5 μg | 5 μg (1 | 5 μg (2 | 5 μg (12 | |
| | | month) | months) | months) | |
| 16 | 10 μg | 10 μg (1 | 10 μg (5 | | |
| | | month) | months) | | |
| 17 | 10 μg | 10 μg (1 | 10 μg (2 | 10 μg (421 | 10 μg (786 |
| | | month) | months) | days) | days) |
| 18 | 5 μg | 5 μg (1 | 5 μg (2 | 5 μg (421 | 5 μg (786 |
| | | month) | months) | days) | days) |

Compositions disclosed herein may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intraarterial, intramuscular, or intrasternal injection, intravenous, or kidney dialytic infusion techniques. Typically, the systemic, parenteral administration is intramuscular injection. As used herein, the term “mucosal administration” includes oral, intranasal, intravaginal, intra-rectal, intra-tracheal, intestinal and ophthalmic administration. Preferably, administration is intramuscular.

In embodiments, a dose is administered in a volume of about 0.1 mL to about 1.5 mL, for example, about 0.1 mL, about 0.2 mL, about 0.25 mL, about 0.3 mL, about 0.4 mL, about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, or about 1.5 mL. In embodiments, the dose is administered in a volume of 0.25 mL. In embodiments, the dose is administered in a volume of 0.5 mL. In embodiments, the dose is administered in a volume of 0.6 mL.

In embodiments, an immunogenic composition may comprise an antigen at a concentration of about 1 μg/mL, to about 50 μg/mL, 10 μg/mL to about 100 μg/mL, about 10 μg/mL to about 50 μg/mL, about 175 μg/mL to about 325 μg/mL, about 200 μg/mL to about 300 μg/mL, about 220 μg/mL, to about 280 μg/mL, or about 240 μg/mL to about 260 μg/mL.

In embodiments, an immunogenic composition described herein has an efficacy of preventing an infection with malaria. The efficacy may be determined by comparing the number of subjects not administered a malaria immunogenic composition that are diagnosed with malaria (controls diagnosed with malaria) to the number of subjects administered a malaria immunogenic composition that are subsequently diagnosed with malaria. The following equation is used to calculate efficacy: 100×(percentage of controls diagnosed with malaria-percentage of subjects administered the immunogenic composition diagnosed with malaria)/(percentage of controls diagnosed with malaria).

In embodiments, the immunogenic compositions described herein have an efficacy of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100%.

In some embodiments, the disclosure provides co-formulation (i.e., prefilled syringes or pre-mix) strategies for immunogenic compositions comprising an antigen of a Plasmodium parasite and an adjuvant (e.g., a saponin adjuvant). Typical vaccine administration strategies currently being utilized are bedside mix formulations. That is, vaccine compositions and adjuvants are stored separately and are mixed prior to administration. Pre-mix, co-formulation, or prefilled syringe strategies for vaccine are less common due to the concerns of the stability of the antigens (e.g., the R21 antigen) and their subsequent immunogenic capabilities. The present disclosure provides immunogenic compositions that can be pre-mixed and stored in advance. The disclosed vaccination strategies and formulations may improve the efficiency of vaccination and may reduce the risks of bedside mixing errors, while maintaining the overall safety and immunogenicity.

A variety of containers may be used to store and transport the pre-mix formulations, including syringes for single administrations and plastic ampules. In some instances, plastic ampules can be manufactured using the blow-fill-seal manufacturing technique or method. In general, the blow-fill-seal (BFS) manufacturing method includes extruding a plastic material (e.g., resin) to form a parison, which is then placed into a mold and cut to size. A filling needle or mandrel is then used to inflate the plastic, which in turn, results in a hollow ampule that substantially conforms to the shape of the mold. Once inflated, a desired volume of liquid can be injected into the ampule, the filling needle or mandrel can be removed, and the ampule can be sealed. Accordingly, BFS can be an automated process that can be performed in a sterile environment without direct human intervention.

In some instances, the ability to aseptically manufacture sterile ampules containing a desired liquid can make BFS manufactured ampules particularly well suited for the pharmaceutical industry. BFS technology, however, has not been compatible with all pharmaceutical liquids, products, etc. For example, some known BFS manufacturing methods include delivering the liquid or product into the ampule while the plastic is still relatively hot, which can result in adverse effects to temperature sensitive liquids and/or products such as vaccines, biologics, etc. Advances in cool BFS technology, however, have increased the variety of suitable products, liquids, etc. allowing some vaccines, biologics, and/or other temperature sensitive pharmaceuticals to be contained in BFS ampules.

In some instances, a BFS ampule can have a size, shape, and/or configuration that is at least partially based on a desired use and/or a desired pharmaceutical liquid or dosage that the ampule is configured to contain. For example, some known BFS ampules can include a pierce through top, a twist-off top, a top including a male or female luer, and/or the like. Some known BFS ampules can have a size and/or shape based on volume of the liquid or dosage configured to be disposed therein. In addition, some known BFS ampules can be manufactured in a strip of multiple, temporarily connected ampules, which can increase manufacturing, packaging, and/or storing efficiencies and/or the like.

In embodiments, the immunogenic compositions described herein are provided in pre-filled syringes. When the immunogenic composition is prepared in a pre-filled syringe, an antigen and adjuvant is combined in advance of administration. In embodiments, the pre-filled syringe contains an antigen of a Plasmodium parasite (e.g., R21) and an adjuvant (e.g., a saponin adjuvant). In embodiments, the pre-filled syringe contains R21 and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant. In embodiments, the pre-filled syringe contains R21 and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant. In embodiments, the pre-filled syringe contains R21 and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina accounts for at least about 75% by weight and fraction C of Quillaja Saponaria Molina accounts for the remainder of the sum of weights of fraction A of Quillaja Saponaria Molina and fraction C of Quillaja Saponaria Molina in the adjuvant.

In embodiments, a subject is administered an immunogenic composition from a pre-filled syringe. In embodiments, the subject is administered an immunogenic composition containing both hemagglutinin and CoV S polypeptide in a single pre-filled syringe. In embodiments, the subject is administered an immunogenic composition from a pre-filled syringe that contains a CoV S polypeptide, but does not contain hemagglutinin. In embodiments, the subject is administered an immunogenic composition from a pre-filled syringe that contains hemagglutinin, but does not contain a CoV S polypeptide.

### EXAMPLES

### Example 1: A Phase I/IIb Randomized, Controlled Trial of Safety and Immunogenicity of an Immunogenic Composition Comprising an R21 Protein and Saponin Adjuvant in 5-17 Month Olds

Purpose: The efficacy of immunogenic compositions comprising R21 protein and saponin adjuvant was evaluated in children aged 5-17 months.

Methods: A Phase I/IIb randomized controlled trial of the safety and immunogenicity of a candidate immunogenic composition was performed in 5-17 month old children in Nanoro, Burkina Faso. Children were randomized into three groups: Group 1, Group 2, and Group 3. Children in Group 1 were administered 5 μg of R21 protein with 25 μg of saponin adjuvant on day 0 (dose 1), day 28 (dose 2), day 56 (dose 3), 1 year after dose 3 or 421 days after dose 1 (dose 4), and 1 year after dose 4 or 786 days after dose 1 (dose 5). Children in Group 2 were administered 5 μg of R21 protein with 50 μg of saponin adjuvant on day 0 (dose 1), day 28 (dose 2), day 56 (dose 3), 1 year after dose 3 or 421 days after dose 1 (dose 4), and 1 year after dose 4 or 786 days after dose 1 (dose 5). Children in group 3 were administered a placebo (control). In each composition, the saponin adjuvant comprised 85% w/w Fraction A ISCOM matrix and 15% w/w Fraction C ISCOM matrix.

Production of R21 protein: The R21 protein was produced in Hansenula polymorpha yeast cells A nucleic acid encoding the R21 protein was subcloned into a derivative of the H. polymorpha expression plasmid pFPMT121 which carried the LEU2 instead URA3 gene for selection in yeast. The auxotrophic H. polymorpha strain ALU3 (relevant genotype: ade1, leu2, ura3) derived from wild type strain ATCCR 34438™ (CBS 4732, IFO 1476, JCM 3621, NBRC 1476, NCYC 1457, NRRL Y-5445) was used as an expression host. Recombinant yeast cell lines were generated by electroporation and a subsequent strain generation and isolation protocol. Thereby, the expression plasmid stably integrated into the yeast genome.

Heterologous yeast strains were stored as glycerol stocks at −80° C. The production cell line was cultured in 2 L baffled shake flasks filled with 200 mL animal component-free YPG medium containing 20 g L⁻¹ glycerol (AppliChem, Germany) as a carbon source and 0.1 g L⁻¹ adenine (AppliChem). Pre-cultures grown in YPD medium to stationary phase were used as inoculum. Cultures were incubated at 37° C. and 130 rpm with 5 cm throw. After 56 h of batch growth and derepression of the promoter system by consumption of glycerol, 1% (v/v) methanol was added to the cultures for induction of target gene expression. After 72 h total cultivation time, cells were harvested by centrifugation (6,000 g, 15 min, 4° C.), washed once with wash buffer (50 mM Na-phosphate buffer, 2 mM EDTA, pH 8.0) and stored at −20° C. The R21 protein was isolated by ultracentrifugation.

Results: No suspected unexpected serious adverse reactions (SUSARs) or serious adverse events (SAEs) related to vaccination were observed. No febrile convulsions were observed. The number of fevers experienced by patients was low across all vaccine doses. 9-15% of patients in Group 1 experienced fevers. 10-30% of patients in Group 2 experienced fevers. 8-10% of patients in Group 3 experienced fevers. Table 1 shows the protective efficacy by group.

TABLE 1
Protective Efficacy % (95% Cl)

| | | | Protective | |
|---|---|---|---|---|
| | Person - years | | Efficacy % | Wald Test |
| Group | at risk | Events | (95% Cl) | P-value |
| 1 | 120.5 | 75 | 69.6 [58.7-77.6] | <0.001 |
| 2 | 121.8 | 60 | 75.7 [66.3-82.4] | <0.001 |
| 3 | 124.0 | 250 | | |

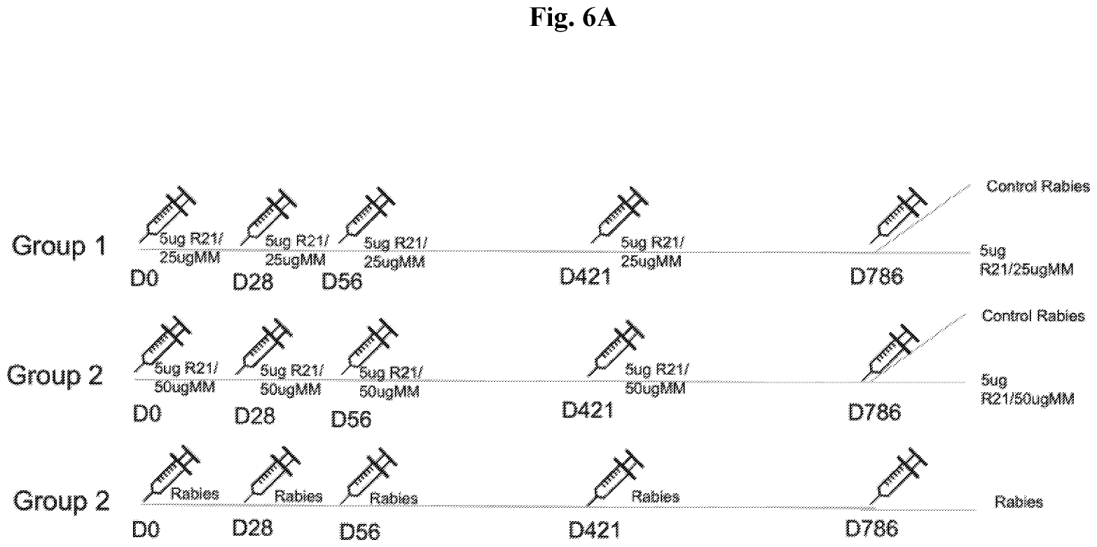

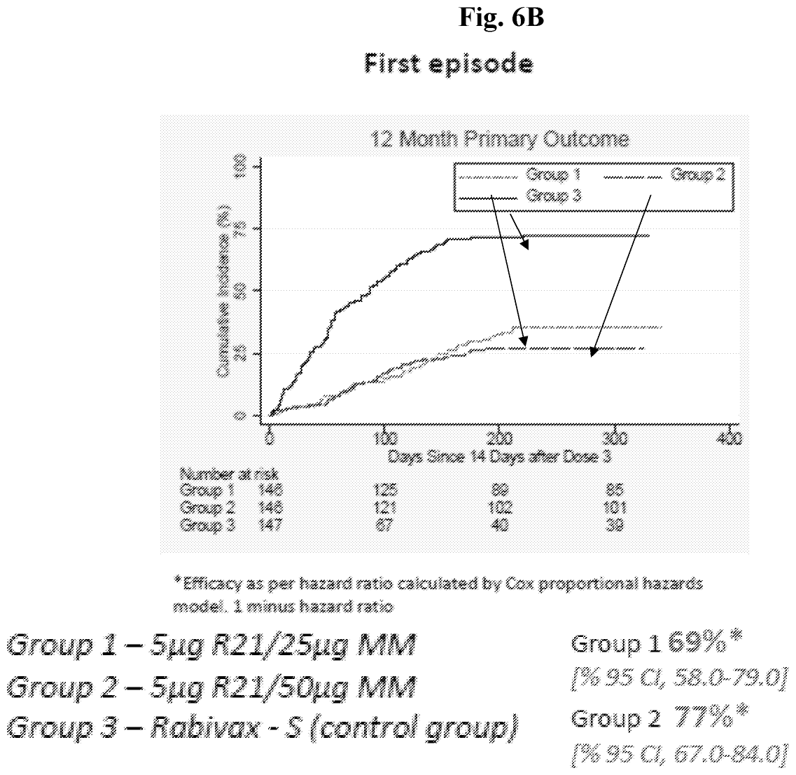

FIG. 6B shows a graph of the incidence of malaria in Group 1, Group 2, and Group 3 patients.

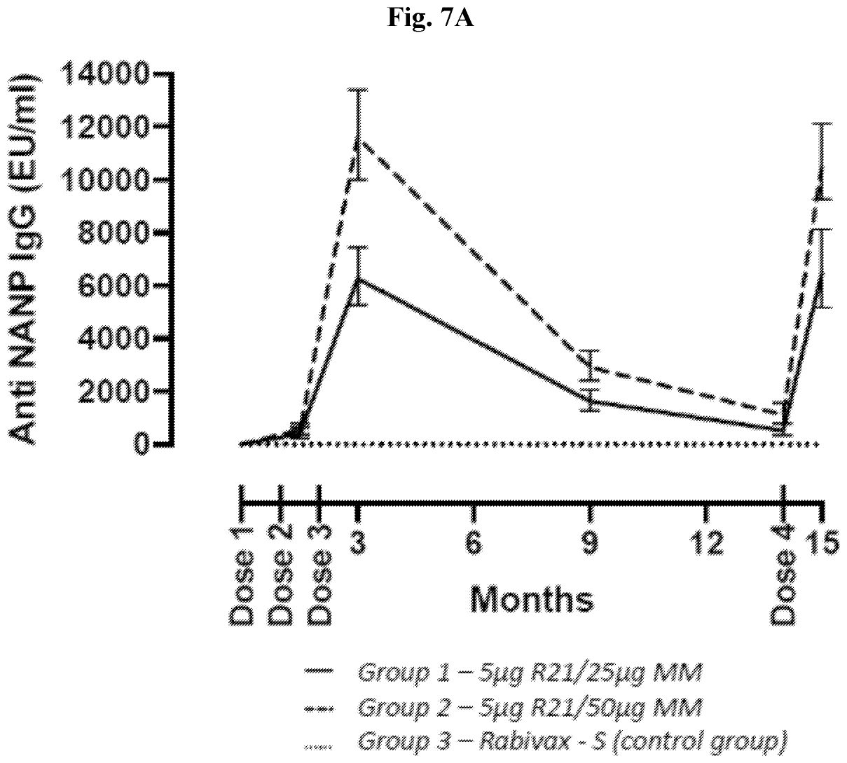

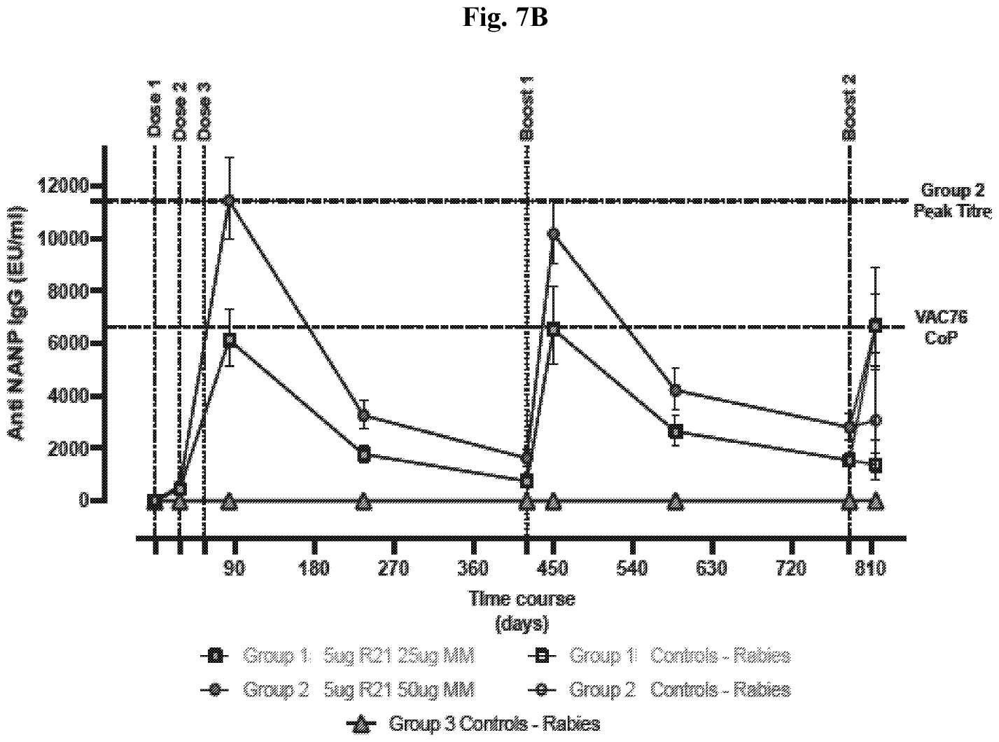

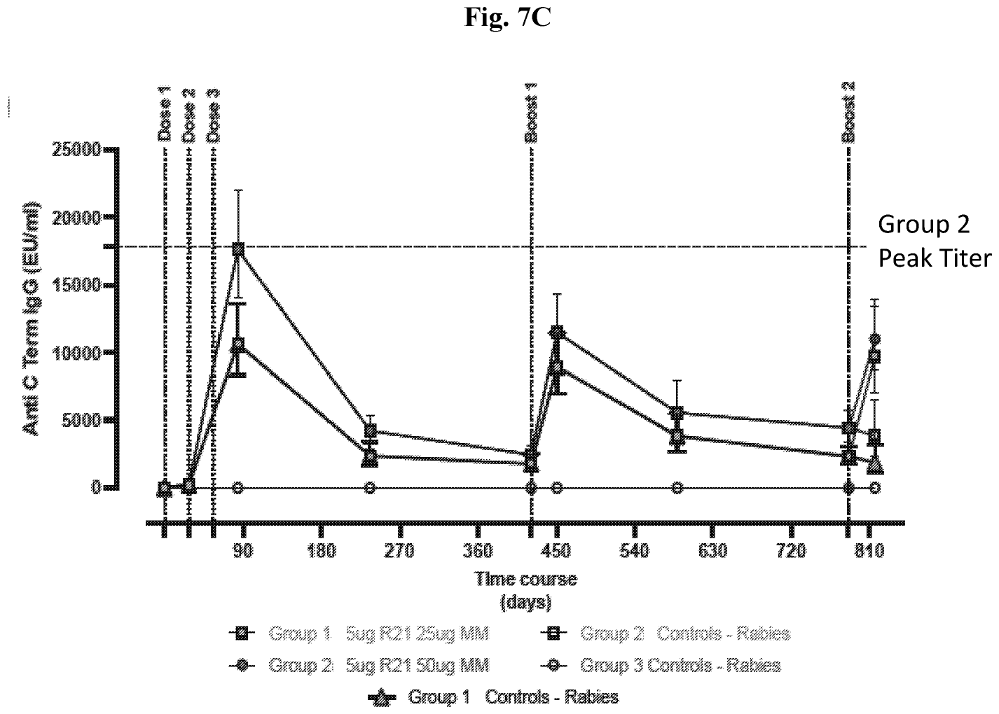

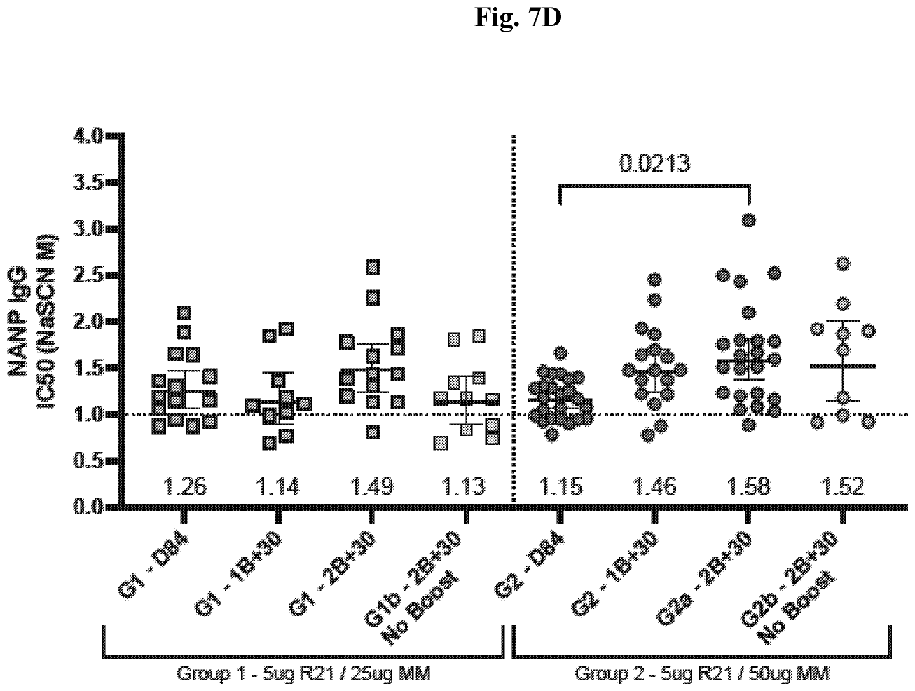

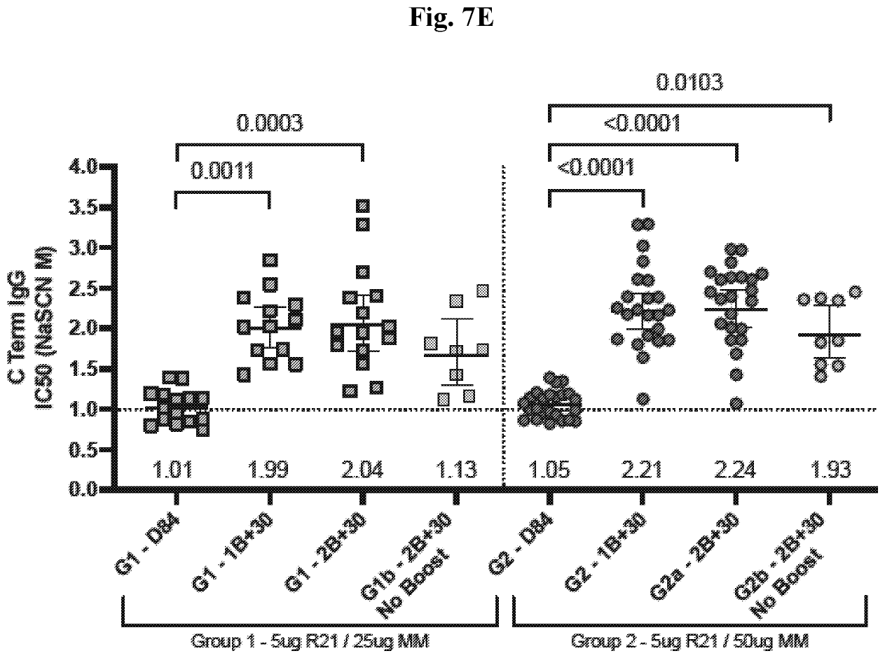

FIG. 7A is a graph showing the immunoglobulins that protect against malaria in patients in Groups 1, 2, and 3. Patients in Group 2 exhibited a higher amount of immunoglobulins after three doses of vaccine than patients in Group 1 (p<0.0001). The patients in both Group 1 and 2 also exhibited an increase in antibody concentration after the boost dose. FIG. 7B is a graph showing the titer of anti-NANP antibodies over time in patients after administration of the aforementioned dosage regimens. FIG. 7C is a graph showing the titer of IgG specific for the C-terminus of the CS protein over time in patients after administration of the aforementioned dosage regimens. FIG. 7D is a graph showing the avidity of the CS protein to anti-NANP antibodies one month after administration of the aforementioned dosage regimens FIG. 7E is a graph showing the avidity of the CS protein to IgG specific for the C-terminus of the CS protein over time in patients after administration of the aforementioned dosage regimens.

Efficacy in the Group 2 patients was 81% [95% Cl: 74-87%] over 12 months post booster assessing time to first clinical malaria episode (p<0.0001). Efficacy in the Group 2 patients was 78% [95% Cl: 71-83%] against multiple episodes of clinical malaria over 12 months post booster (p<0.001). Efficacy in the Group 2 patients was 77% [95% CI: 71-83%] against multiple episodes of clinical malaria over 24 months following a primary series of vaccinations (p<0.001).

### Example 2A: A Phase I/IIa Open-Label Trial of Safety and Immunogenicity of an Immunogenic Composition Comprising an R21 Protein and Saponin Adjuvant in Adults

Purpose: The efficacy of immunogenic compositions comprising R21 protein and saponin adjuvant was evaluated in adults.

Methods: A Phase I/IIa non-randomized open-label trial of the safety and immunogenicity of a candidate immunogenic composition was performed in adults in Oxford, London, and Southampton, England. Group 2 was administered 10 μg R21 protein and 50 μg of saponin adjuvant (i.e. a saponin adjuvant comprising 85% w/w Fraction A ISCOM matrix and 15% w/w Fraction C ISCOM matrix) at 0 months, 1 months, and 6 months. Group 3 was administered 10 μg R21 protein and 50 μg of saponin adjuvant (i.e. a saponin adjuvant comprising 85% w/w Fraction A ISCOM matrix and 15% w/w Fraction C ISCOM matrix) at 0 months, 1 months, and 2 months. Group 6 was a control group that was not administered a vaccine. The efficacy for each group was calculated on day 21 using the equation: 100×(percentage of subjects not administered an immunogenic composition that are diagnosed with malaria-percentage of subjects diagnosed with malaria that are administered an immunogenic composition)/(percentage of subjects not administered an immunogenic composition that are diagnosed with malaria). The subjects were deliberately infected with malaria parasites (CHMI). The percentage of patients undiagnosed with malaria after CHMI was measured.

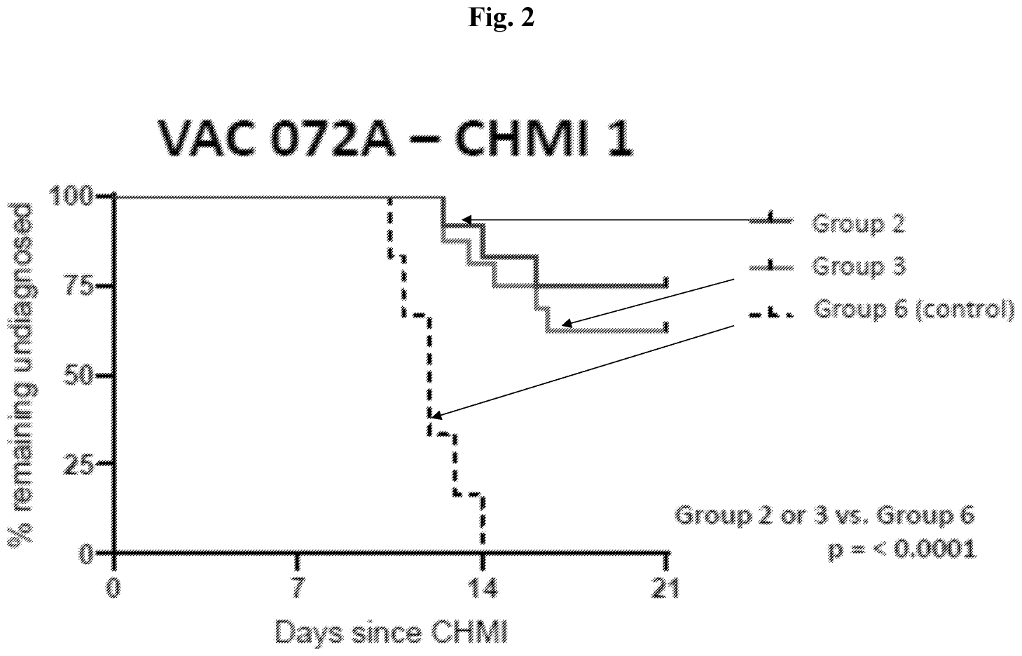

Results: FIG. 2 is a graph showing the percentage of subjects over time that are undiagnosed with malaria after the subjects are deliberately infected with human malaria (CHMI). The efficacy for group 2 was 75%. The efficacy for group 3 was 62.5%. The percentage of controls diagnosed with malaria was 100%.

### Example 2B: A Phase I/IIa Open-Label Trial of Safety and Immunogenicity of an Immunogenic Composition Comprising an R21 Protein and Saponin Adjuvant in Adults

Purpose: The efficacy of immunogenic compositions comprising R21 protein and saponin adjuvant were evaluated in adults.

Methods: A Phase I/IIa non-randomized open-label trial of the safety and immunogenicity of a candidate immunogenic composition was performed in adults in Oxford, London, and Southampton, England. Group 2 was administered 10 μg R21 protein and 50 μg of saponin adjuvant (i.e. a saponin adjuvant comprising 85% w/w Fraction A ISCOM matrix and 15% w/w Fraction C ISCOM matrix) at 0 months, 1 months, 6 months, and 14 months. Group 3 was administered 10 μg R21 protein and 50 μg of saponin adjuvant (i.e. a saponin adjuvant comprising 85% w/w Fraction A ISCOM matrix and 15% w/w Fraction C ISCOM matrix) at 0 months, 1, 2, and 10 months. Group 4 was administered 10 μg R21 protein and 50 μg of saponin adjuvant (i.e. a saponin adjuvant comprising 85% w/w Fraction A ISCOM matrix and 15% w/w Fraction C ISCOM matrix) at 0 months, 1 months, and 6 months. Group 7 was a control group that was not administered vaccinations. VE was calculated on day 14 as in Example 2A. The subjects were deliberately infected with malaria parasites (CHMI). The percentage of patients undiagnosed with malaria after CHMI was measured.

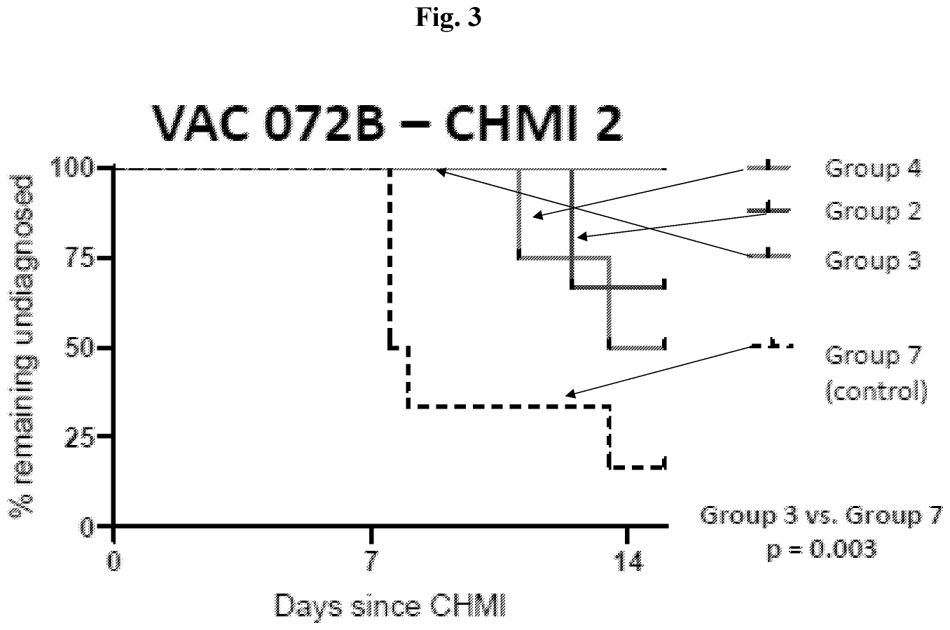

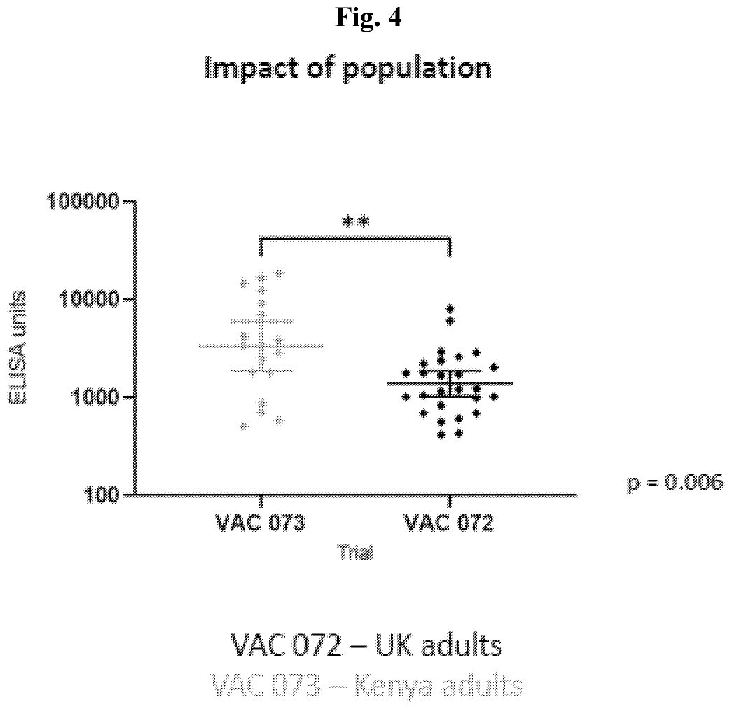

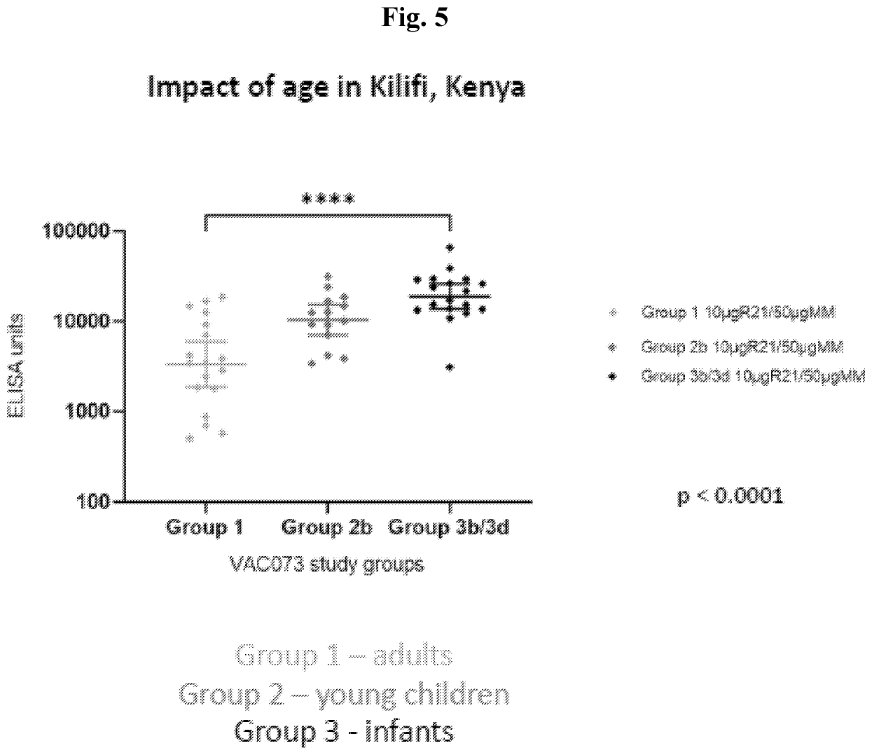

Results: FIG. 3 is a graph showing the percentage of subjects over time that are undiagnosed with malaria after the subjects are deliberately infected with human malaria (CHMI). Group 2 exhibited 60% VE. Group 3 exhibited 100% VE. Group 4 exhibited 40% VE. The percentage of controls diagnosed with malaria was 83.3%.

### Example 3: A Phase III Randomized, Controlled Trial to Evaluate the Efficacy of an Immunogenic Composition Comprising an R21 Protein and Saponin Adjuvant in 5-36 Month Old Children

Purpose: The efficacy of immunogenic compositions comprising R21 protein and saponin adjuvant is being evaluated in children aged 5-36 months.

Methods: A Phase III randomized trial evaluating the efficacy of a candidate immunogenic composition against malaria in 5-36 month old African children is being performed. All children were administered 5 μg of R21 protein with 50 μg of saponin adjuvant or a control vaccine on days 0, 28, and 56. Children in Dande, Bagamoyo, Kilfi are administered a boost on day 421 Children in Nanoro, Bougouni are administered a boost dose after one year. The primary vaccination series is administered prior to or at the start of malaria season.

The following outcomes are evaluated: presence of primary, secondary or tertiary clinical malaria; the safety and reactivity of the immunogenic compositions; solicited adverse events from the composition; and efficacy against clinical malaria. The immunogenicity of the immunogenic composition described herein is compared to a control. Enzyme linked immunosorbent assay (ELISA) is used to quantify antibodies against the CS antigen, the NANP repeat region of CS, and antibodies against HBsAg.

### Example 4: Immunogenicity of Different Dose Regimens in Malaria Naïve Patients Administered an Immunogenic Composition Comprising a R21 Protein and a Saponin Adjuvant

Purpose: The effect of different dose regimens of immunogenic compositions comprising a R21 protein and a saponin adjuvant on immunogenicity was evaluated. The saponin adjuvant comprised 85% w/w Fraction A ISCOM matrix and 15% w/w Fraction C ISCOM matrix. After administration of a dose regimen of Table 2, patients were given a controlled human malaria infection (CHMI).

The regimens are described in Table 2:

TABLE 2
Dose Regimen

| | | Dose 2 | Dose 3 | CHMI |
|---|---|---|---|---|
| | Dose 1 | (28 days after | (days after | (day safter |
| Group | (day 0) | Dose 1) | first dose) | Dose 1) |
| 1 | 10 μg R21 | 10 μg R21 | 10 μg R21 | n/a* |
| | protein; 50 μg | protein; 50 μg | protein; 50 μg | |
| | of saponin | of saponin | of saponin | |
| | adjμvant | adjμvant | adjμvant (56 | |
| | | | days) | |
| 2 | 10 μg R21 | 10 μg R21 | 10 μg R21 | 196 days |
| | protein; 50 μg | protein; 50 μg | protein; 50 μg | |
| | of saponin | of saponin | of saponin | |
| | adjμvant | adjμvant | adjμvant (168 | |
| | | | days) | |
| 3 | 10 μg R21 | 10 μg R21 | 10 μg R21 | 84 days |
| | protein; 50 μg | protein; 50 μg | protein; 50 μg | |
| | of saponin | of saponin | of saponin | |
| | adjμvant | adjμvant | adjμvant (56 | |
| | | | days) | |
| 4 | 50 μg R21 | 50 μg R21 | 10 μg R21 | 196 days |
| | protein; 50 μg | protein; 50 μg | protein; 50 μg | |
| | of saponin | of saponin | of saponin | |
| | adjμvant | adjμvant | adjμvant (168 | |
| | | | days) | |
| 5 | 10 μg R21 | 10 μg R21 | 2 μg R21 | 196 days |
| | protein; 50 μg | protein; 50 μg | protein; 50 μg | |
| | of saponin | of saponin | of saponin | |
| | adjμvant | adjμvant | adjμvant (168 | |
| | | | days) | |

*Patients in Group 1 are not given a CHMI.

Patients in Group 1 were not administered a CHMI and served as a control group. Patients in Group 2 were administered a delayed third dose administered 5 months after the first dose. Patients in Group 3 were administered each dose about 1 month apart. Patients in Group 4 were administered a 50 μg R21 protein at doses 1 and 2 and a dose that is one fifth of dose 1 and 2 (10 μg R21 protein) at dose 3. Patients in Group 5 were administered a 10 μg R21 protein at doses 1 and 2 and a dose that was one fifth of dose 1 and 2 (2 μg R21 protein) at dose 3.

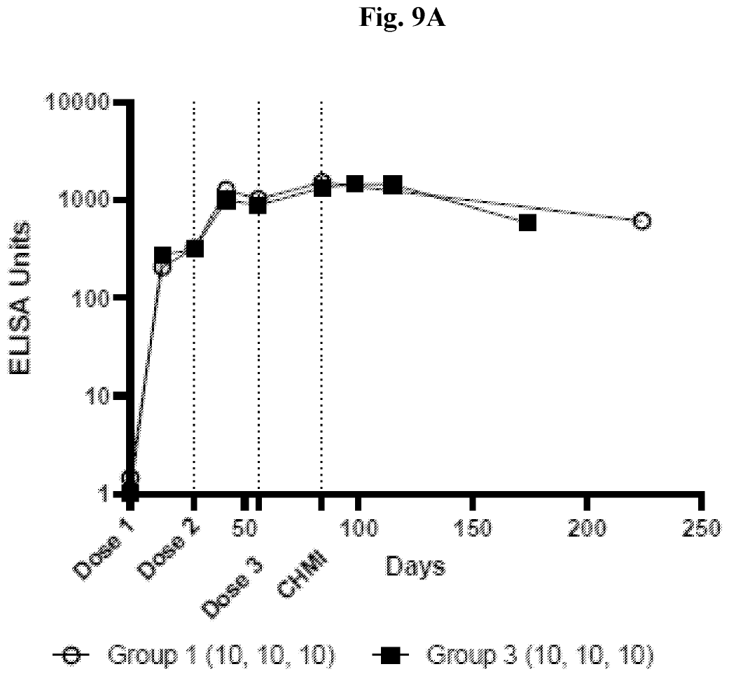

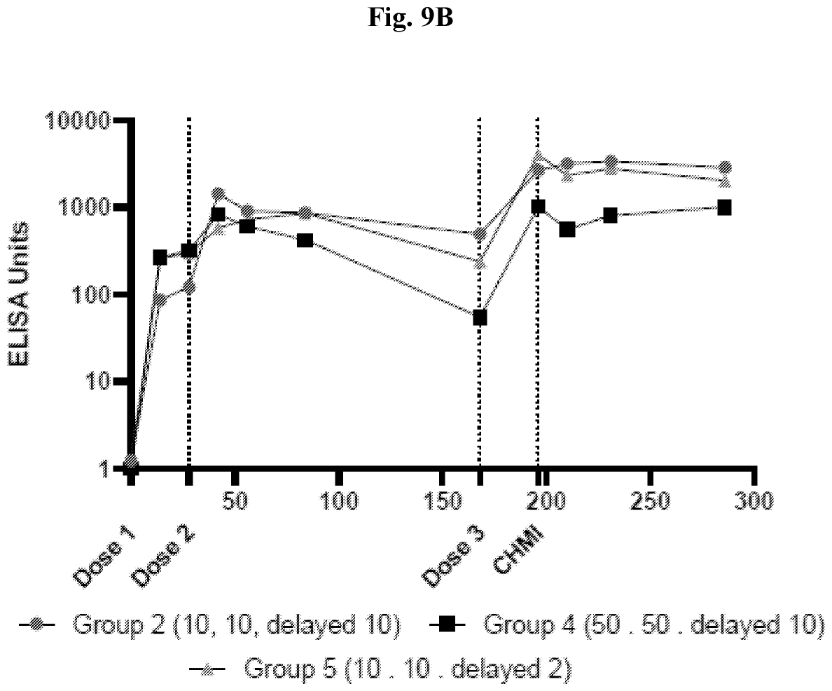

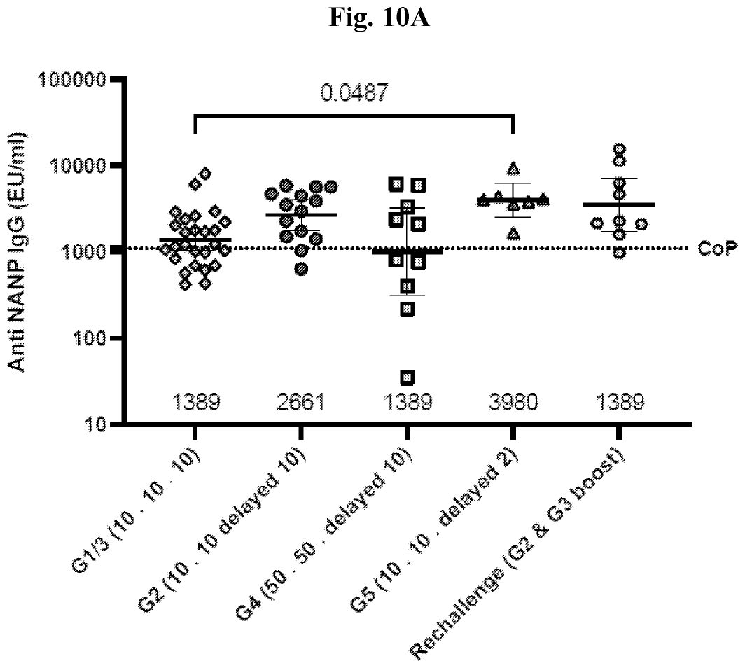

The total IgG NANP-specific antibody response to the central repeat region of the CS protein was evaluated by ELISA. FIGS. 9A and 9B show the antibody response induced over time in patients in groups 1 and 3 (FIG. 9A) versus groups 2, 4, and 5. The patients in groups 2, 4, and 5 were administered a delayed third dose about 3 months after administration of the first dose. Surprisingly and unexpectedly, patients in Group 5 who were administered 2 μg of R21 in dose 3 exhibited about the same antibody induction as patients administered the Group 2 dose regimen (10 μg of R21 in doses 1-3). The antibody response of patients in Group 5 was higher than the antibody response of patients administered 50 μg R21 at dose 1, 50 μg R21 at dose 2, and 10 μg R21 at dose 3.

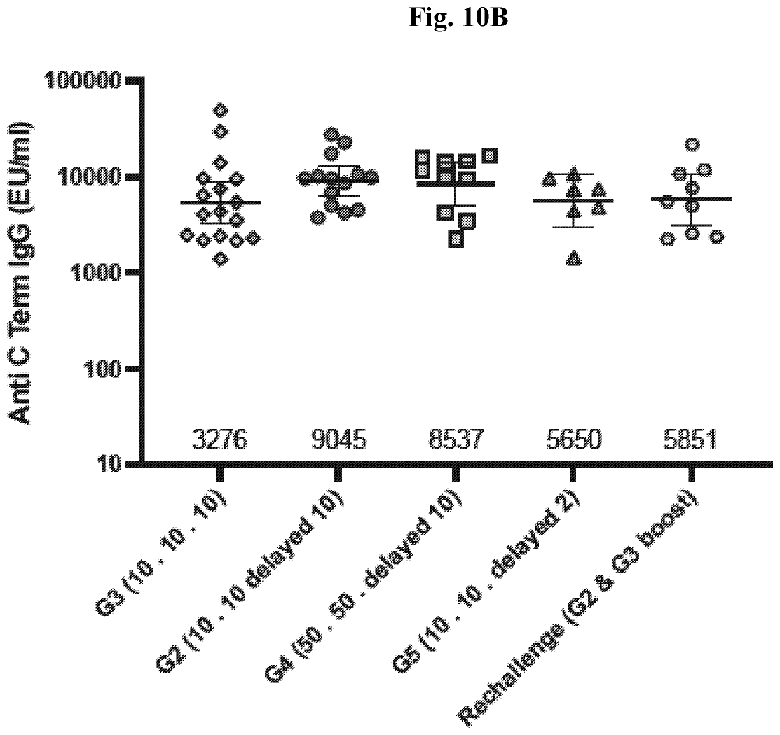

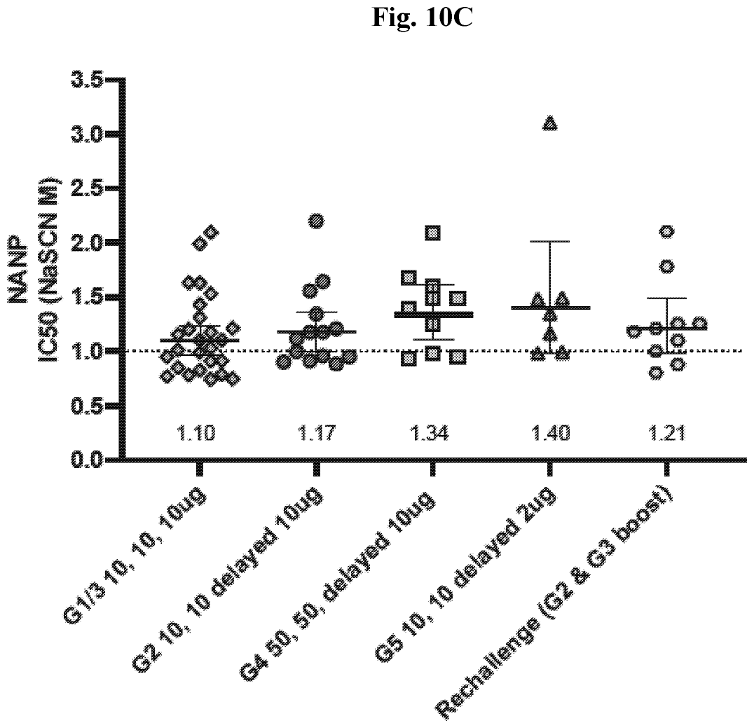

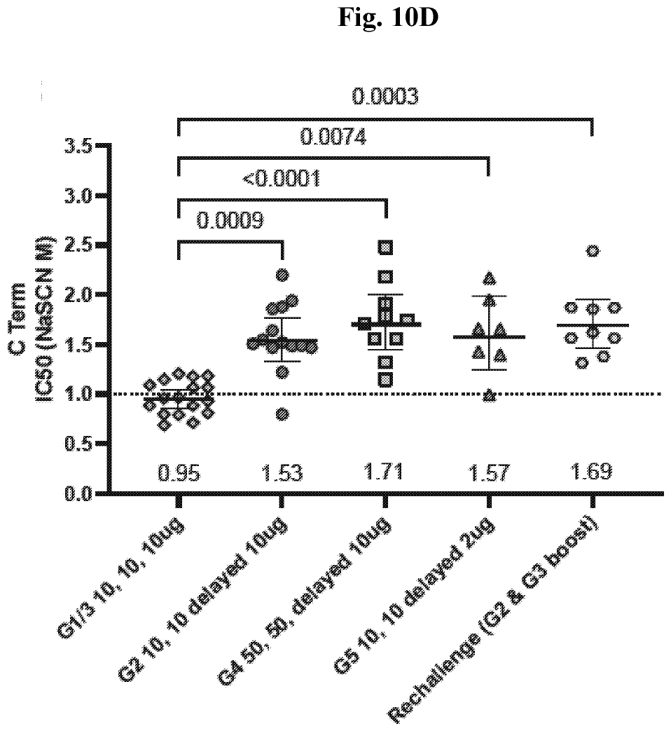

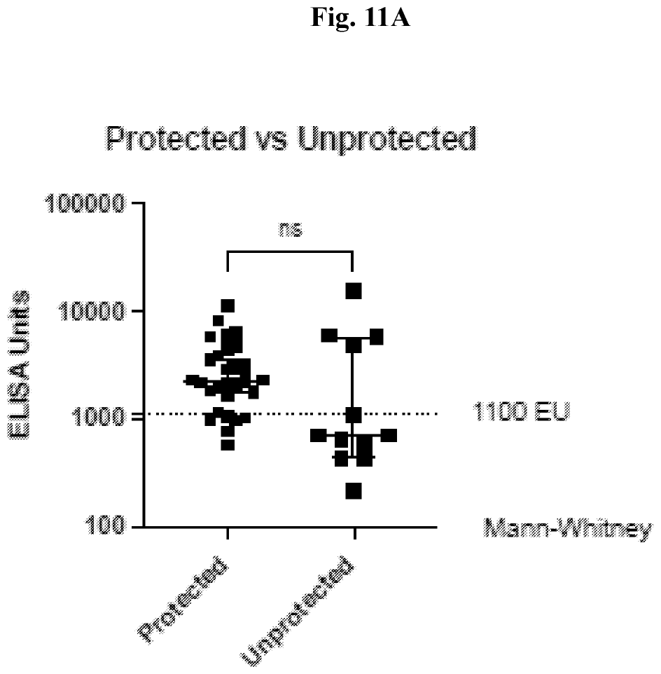

FIG. 10A shows the antibody response (anti-NANP specific IgG) after administration of different dosage regimens of Table 2. Patients administered the regimen of Group 5 exhibit a peak antibody response that is greater than patients administered the regimen of Group 3. The geometric mean of ELISA units in FIG. 10A for Group 3 is 1389 (95% CI 1031-1871). The geometric mean of ELISA units in FIG. 10A for Group 2 is 2661 (95% CI 1771-3998). The geometric mean of ELISA units in FIG. 10A for Group 4 is 1009 (95% CI 316-3221). The geometric mean of ELISA units in FIG. 10A for Group 5 is 3980 (95% CI 2500-6337). FIG. 10B shows the antibody response (IgG specific for the C terminus of the CS protein) after administration of different dosage regimens of Table 2. FIG. 10C shows the avidity of anti-NANP specific IgG for the CS protein after administration of different dosage regimens of Table. FIG. 10D shows the avidity of IgG specific for the C terminus of the CS protein after administration of different dosage regimens of Table 2.

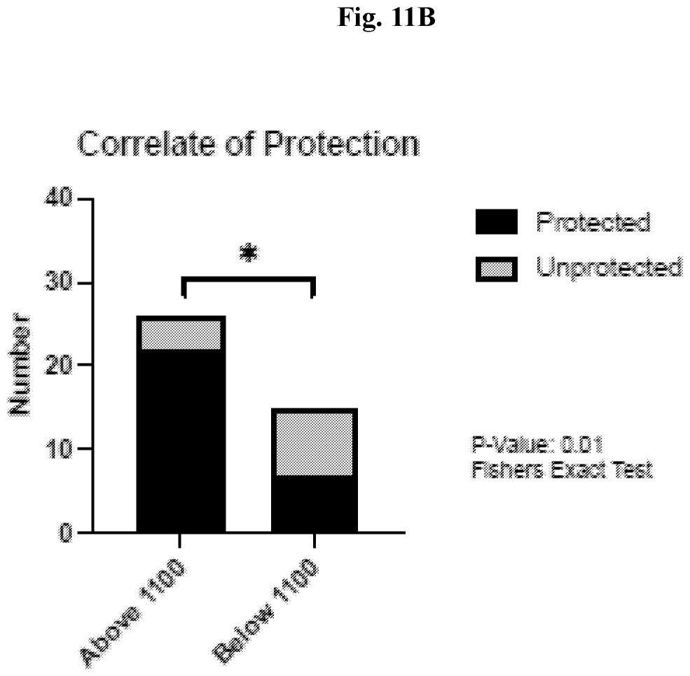

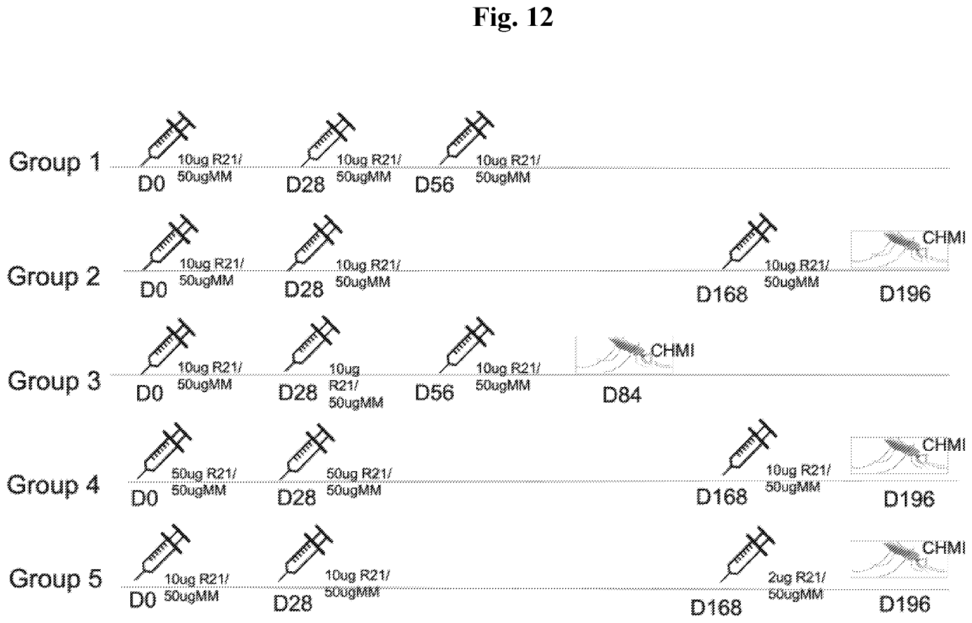

FIG. 11A show antibody titers from patients administered a dose regimen of Group 2 and 3 stratified by protection and unprotection. FIG. 11B shows a correlate of protection in patients of Group 2 and Group 3 with an antibody response EU>1100 or EU<1100.

Collectively, this data shows that a delayed third dose (administered about ˜3 months after the first dose) elicits a greater antibody response than a dosage regimen in which the immunogenic composition is administered in three doses separated by about one month apart.

This data also shows that patients administered a delayed third dose of R21 that is ⅕ lower than the first dose induces a superior antibody response compared to the dose regimen in which 10 μg of R21 is administered in three doses separated by about one month apart. This has significant economic implications because it will reduce the cost of administering immunogenic compositions against Plasmodium parasites (e.g., malaria vaccines).

### NUMBERED EMBODIMENTS

1. An immunogenic composition comprising an antigen of a Plasmodium parasite.
2. The immunogenic composition of embodiment 1, wherein the Plasmodium parasite is Plasmodium falciparum, Plasmodium malariae, Plasmodium vivax, Plasmodium ovale, or Plasmodium knowlesi.
3. The immunogenic composition of embodiment 1 or 2, wherein the antigen comprises a circumsporozoite (CS) protein or a fragment thereof.
4. The immunogenic composition of embodiment 3, wherein the CS protein is from Plasmodium falciparum.
5. The immunogenic composition of any one of embodiments 1-4, wherein the antigen comprises a hepatitis B surface antigen (HBsAg) or fragment thereof.
6. The immunogenic composition of any one of embodiments 1-5, wherein the antigen comprises an amino acid sequence of SEQ ID NO: 1 or an amino acid sequence that is at least 80%, at least 85%, at least 90%, at least 95%, at least 96%, at least 97%, at least 98%, at least 99%, or 100% identical to SEQ ID NO: 1.
7. The immunogenic composition of embodiment 1, comprising an adjuvant.
8. The immunogenic composition of embodiment 7, wherein the adjuvant comprises at least two iscom particles, wherein:
   the first iscom particle comprises fraction A of Quillaja Saponaria Molina and not fraction C of Quillaja Saponaria Molina; and
   the second iscom particle comprises fraction C of Quillaja Saponaria Molina and not fraction A of Quillaja Saponaria Molina.
9. The immunogenic composition of embodiment 8, wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant.
10. The immunogenic composition of embodiment 8, wherein fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant.
11. The immunogenic composition of any one of embodiments 7-10, wherein the adjuvant is administered at a dose of about 50 μg.
12. A method of stimulating an immune response against a Plasmodium parasite in a subject comprising administering the immunogenic composition of any one of embodiments 1-11.
13. The method of embodiment 12, wherein the immunogenic composition comprises an adjuvant.
14. The method of embodiment 13, wherein the adjuvant comprises at least two iscom particles, wherein:
   the first iscom particle comprises fraction A of Quillaja Saponaria Molina and not fraction C of Quillaja Saponaria Molina; and
   the second iscom particle comprises fraction C of Quillaja Saponaria Molina and not fraction A of Quillaja Saponaria Molina.
15. The method of embodiment 14, wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant.
16. The method of embodiment 14, wherein fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant.
17. The method of any one of embodiments 13-16, wherein the adjuvant is administered at a dose of about 50 μg.
18. The method of any one of embodiments 13-16, wherein the dose of antigen administered is from about 0.1 μg to about 100 μg.
19. The method of any one of embodiments 13-16, wherein the dose of antigen administered is from about 0.1 μg to about 10 μg.
20. The method of any one of embodiments 13-16, wherein the dose of antigen administered is from about 0.1 μg to about 5 μg.
21. The method of any one of embodiments 13-16, wherein the dose of antigen administered is from about 0.1 μg to about 3 μg.
22. The method of any one of embodiments 13-16, wherein amount of antigen administered is from about 0.1 μg to about 2 μg.
23. The method of any one of embodiments 12-22, comprising administering a first dose and a second dose of the immunogenic composition.
24. The method of embodiment 23 comprising administering a third dose of the immunogenic composition.
25. The method of embodiment 23, wherein the dose of antigen in the second dose is less than the dose of antigen in the first dose.
26. The method of embodiment 24, wherein the dose of antigen in the third dose is less than the dose of antigen in the first dose.
27. The method of embodiment 23 or 24, wherein the first dose and second dose comprise the same amount of antigen.
28. The method of embodiment 25, wherein the second dose and third dose comprise the same amount of antigen.
29 The method of embodiment 25, wherein the first and third dose comprise the same amount of antigen.
30. The method of embodiment 26, wherein the third dose comprises about 90%, about 80%, about 70%, about 60%, about 50%, about 40%, about 30%, about 20%, or about 10% of the antigen in the first dose.
31. The method of embodiment 26, wherein the second dose comprises about 90%, about 80%, about 70%, about 60%, about 50%, about 40%, about 30%, about 20%, or about 10% of the antigen in the first dose.
32. The method of embodiment 23 or 24, wherein the second dose is administered about 28 days after the first dose.
33. The method of embodiment 24, wherein the third dose is administered about 56 days after the first dose.
34. The method of embodiment 24, wherein the third dose is administered about 168 days after the first dose.
35. A method of stimulating an immune response against a Plasmodium parasite in a subject comprising administering:
   (i) a first dose of an immunogenic composition comprising an antigen of a Plasmodium parasite; wherein the first dose comprises about 10 μg of the antigen;
   (ii) a second dose of the immunogenic composition, wherein the second dose comprises about 10 μg of the antigen; and
   (iii) a third dose of the immunogenic composition, wherein the third dose comprises about 2 μg of the antigen.
36. The method of embodiment 35, wherein the second dose is administered about 28 days after the first dose.
37. The method of embodiment 35 or 36, wherein the third dose is administered about 168 days after the first dose.
38. The method of any one of embodiments 35-37, wherein the Plasmodium parasite is Plasmodium falciparum, Plasmodium malariae, Plasmodium vivax, Plasmodium ovale, or Plasmodium knowlesi.
39 The method of any one of embodiments 35-38, wherein the antigen comprises a circumsporozoite (CS) protein or a fragment thereof.
40. The method of any one of embodiments 35-39, wherein the CS protein is from Plasmodium falciparum.
41. The method of any one of 35-40, wherein the antigen comprises a a hepatitis B surface antigen (HBsAg) or fragment thereof.
42. The method of any one of embodiments 35-41, wherein the antigen comprises an amino acid sequence of SEQ ID NO: 1 or an amino acid sequence that is at least 80%, at least 85%, at least 90%, at least 95%, at least 96%, at least 97%, at least 98%, at least 99%, or 100% identical to SEQ ID NO: 1.
43. The method of any one of embodiments 35-42, comprising an adjuvant.
44. The method of embodiment 43, wherein the adjuvant comprises at least two iscom particles, wherein:
   the first iscom particle comprises fraction A of Quillaja Saponaria Molina and not fraction C of Quillaja Saponaria Molina; and
   the second iscom particle comprises fraction C of Quillaja Saponaria Molina and not fraction A of Quillaja Saponaria Molina.
45. The method of embodiment 44, wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant.
46. The method of embodiment 44, wherein fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant.
47. The method of any one of embodiments 35-46, wherein the adjuvant is administered at a dose of about 50 μg.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. This application hereby incorporates by reference the disclosures of U.S. Pat. Nos. 10,729,764, 9,821,046, and 8,821,881 in their entireties for all purposes.

1-69. (canceled)

70. A method of stimulating an immune response against a Plasmodium parasite in a subject comprising administering an immunogenic composition comprising an antigen of a Plasmodium parasite, wherein the antigen comprises a circumsporozoite (CS) protein or a fragment thereof and a hepatitis B surface antigen (HBsAg) or a fragment thereof.

71. The method of claim 70, wherein the antigen of the Plasmodium parasite comprises the amino acid sequence of SEQ ID NO: 1 or SEQ ID NO: 2 or an amino acid sequence with at least 80%, at least 85%, at least 90%, at least 91%, at least 92%, at least 93%, at least 94%, at least 95%, at least 96%, at least 97%, at least 98%, at least 99%, or at least 99.5% identity to SEQ ID NO: 1 or SEQ ID NO: 2.

72. The method of claim 70, wherein the immunogenic composition comprises an adjuvant.

73. The method of claim 72, wherein the immunogenic composition comprises from about 1 μg to about 100 μg of adjuvant.

74. The method of claim 73, wherein the immunogenic composition comprises from about 25 μg to about 75 μg of adjuvant.

75. The method of claim 72, wherein the adjuvant comprises at least two iscom particles, wherein:
the first iscom particle comprises fraction A of Quillaja Saponaria Molina and not fraction C of Quillaja Saponaria Molina; and
the second iscom particle comprises fraction C of Quillaja Saponaria Molina and not fraction A of Quillaja Saponaria Molina.

76. The method of claim 75, wherein fraction A of Quillaja Saponaria Molina accounts for at least 75% by weight and fraction C of Quillaja Saponaria Molina accounts for the remainder, respectively, of the sum of the weights of fraction A of Quillaja Saponaria Molina and fraction C of Quillaja Saponaria Molina in the adjuvant.

77. The method of claim 70, comprising administering the immunogenic composition in a prefilled syringe.

78. The method of claim 70, wherein the dose of antigen administered is from about 0.1 μg to about 100 μg; from about 0.1 μg to about 10 μg; from about 0.1 μg to about 5 μg; from about 0.1 μg to about 3 μg; or from about 0.1 μg to about 2 μg.

79. The method of claim 70, comprising administering a first dose and a second dose of the immunogenic composition.

80. The method of claim 79, comprising administering a third dose of the immunogenic composition.

81. The method of claim 79, wherein the dose of antigen in the second dose is less than the dose of antigen in the first dose.

82. The method of claim 79, wherein the first dose and second dose comprise about the same amount of antigen.

83. The method of claim 79, comprising administering the second dose about 1 month after the first dose.

84. The method of claim 79, comprising administering a fourth dose, fifth dose, sixth dose, seventh dose, eighth dose, ninth dose, or tenth dose of the immunogenic composition

85. The method of claim 1, wherein the Plasmodium parasite is Plasmodium falciparum, Plasmodium malariae, Plasmodium vivax, Plasmodium ovale, or Plasmodium knowlesi.

86. The method of claim 70, wherein the CS protein is from Plasmodium falciparum.

87. The method of claim 70, wherein the method prevents malaria with an efficacy from about 50% to about 99%, from about 50% to about 95%, from about 50% to about 90%, from about 50% to about 85%, from about 50% to about 80%, from about 60% to about 99%, from about 65% to about 95%, from about 65% to about 90%, from about 65% to about 85%, from about 69% to about 81%, from about 60% to about 95%, from about 60% to about 90%, from about 60% to about 85%, from about 60% to about 80%, from about 40% to about 99%, from about 40% to about 95%, from about 40% to about 90%, from about 40% to about 85%, from about 40% to about 80%, from about 40% to about 75%, from about 40% to about 70%, from about 40% to about 65%, from about 40% to about 55%, or from about 40% to about 50% for up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, up to about 12 months, up to 13 months, up to 14 months, up to 15 months, up to 16 months, up to 17 months, up to 18 months, up to 19 months, up to 20 months, up to 21 months, up to 22 months, up to 23 months, or up to 24 months after administration of the immunogenic composition.

88. The method of claim 70, wherein the method prevents malaria with an efficacy of about 69% for at least about 12 months.

89. The method of claim 70, wherein the method prevents malaria with an efficacy of at least about 77% for at least about 24 months.
